(19) United States
(12) Patent Application Publication
(10) Pub. No.: US 2017/0043156 A1
(43) Pub. Date: Feb. 16, 2017

(54) SYSTEM AND METHOD FOR IMPLANTATION OF LEAD AND ELECTRODES TO THE ENDOPELVIC PORTION OF THE PELVIC NERVES AND CONNECTION CABLE FOR ELECTRODE WITH DIRECTION MARKER

(71) Applicant: Marc Possover, Koln (DE)

(72) Inventor: Marc Possover, Koln (DE)

(21) Appl. No.: 14/826,489

(22) Filed: Aug. 14, 2015

(51) Int. Cl. A61N 1/05; A61N 1/36

(52) U.S. Cl. CPC A61N 1/0551; A61N 1/36007; A61N 1/36071; A61N 1/36107

(57) ABSTRACT

A collector electrode assembly which can be implanted by laparoscopy through the abdominal wall into the small pelvis of the human body includes a collector electrode for neurostimulation of nerves; a connection cable having an outer surface, said collector electrode being arranged at one end of said connection cable and comprising several outer segment electrodes which can be contacted individually and/or in groups and which are arranged axially one after another in the direction of the longitudinal extent of the collector electrode, wherein an insulating section is arranged axially between in each case two adjacent outer segment electrodes and permits electrical insulation of respective two adjacent outer segment electrodes; radially expandable fixing structures positioned on the collector electrode and radially expandable from a withdrawn position to a radially expanded position for fixing the collector electrode in place at said nerves; a visually perceptible direction marker on the outer surface of the connection cable, at least in a cable section which is spaced apart from axial ends of the connection cable and has an axial extent of at least 10 cm and/or at least 15% of total length of the connection cable, said direction marker indicating orientation of the connection cable to an operator using the assembly, and wherein the direction marker is designed and arranged in such a way that the identification of the orientation of the connection cable is possible at any desired axial section of the cable section having a maximum axial extent of 2 cm.

### BACKGROUND OF THE INVENTION

The invention relates to a tool, system and method for treating at least one symptom of a pelvic floor and organ disorder and neuropathic pain by implanting a lead and electrode to the endopelvic portion of the pelvic nerves, nerves roots and/or plexuses using the tool, system and method according to the invention.

Pelvic floor disorders adversely affect the health and quality of life of millions of people. Pelvic floor disorders include urinary control disorders such as urge incontinency, urge frequency, voiding efficiency, fecal control disorders, sexual dysfunctions, and pelvic pain.

Lower urinary tract disorders affect the quality of life of millions of men and women over the world every year.

Thirteen million Americans suffer from various types of urinary incontinence (UI). The most prevalent type of UI (22% of the total) is called Stress Incontinence (SUI). SUI is characterized by the unintended emission of urine during everyday activities and events, such as laughing, coughing, sneezing, exercising, or lifting. These activities and events cause an increase in bladder pressure resulting in loss of urine due to inadequate contraction of the sphincter muscle around the outlet of the bladder.

Another prevalent type of such urinary disorder is the urinary urge incontinence (18% of the total) that is characterized by a strong desire to urinate, followed by involuntary contractions of the bladder. Such disorders of the lower urinary tract include overactive bladder, interstitial cystitis, prostatis, prostadynia and benign prostatic hyperplasia.

Many people (47% of the total) encounter a combination of bladder control disorders.

Overactive bladder (OAB) is a medical condition estimated to affect 17 to 20 million people in the United States. Symptoms of OAB can include urinary frequency, urinary urgency, urinary urge incontinence due to a sudden and unstoppable need to urinate, nocturia or enuresis resulting from over activity of the detrusor muscle.

Neurogenic OAB occurs as a result of detrusor muscle over activity referred to as detrusor hyperreflexia, secondary to known neurologic disorders, such as stroke, Parkinson's disease, diabetes, multiple sclerosis, peripheral neuropathies, or spinal cord injuries. In contrast, non-neurogenic OAB occurs as a result of detrusor muscle over activity referred to as detrusor muscle instability that arises from non-neurological abnormalities, such as bladder stones, muscle disease, urinary tract infection or drug side effects, or can be idiopathic (the most frequent situation).

Interstitial cystitis (IC) is another lower urinary tract disorder of unknown etiology that predominantly affects young and middle-aged females, although men and children can also be affected. Symptoms of IC can include irritative voiding symptoms, urinary frequency, urinary urgency, nocturia or suprapubic or pelvic pain related to and relieved by voiding. Many IC patients also experience headaches as well as gastrointestinal and skin problems. In some cases, IC can also be associated with ulcers or scars of the bladder.

Prostatitis and prostadynia are other lower urinary tract disorders that have been suggested to affect approximately 2% of the adult male population (Collins M M et al., How common is prostatitis? A national survey of physician visits. J Urol 1998; 159:1224-1228). Prostatitis is an inflammation of the prostate, and includes bacterial prostatitis and non-bacterial etiologies. Chronic non-bacterial prostatitis is distinguished from acute bacterial prostatitis based on the recurrent nature of the disorder.

Most patients affected by pelvic floor disorders not only suffer from urinary but also from intestinal disorders, and mostly from both together. Fecal incontinence and constipation are the most frequent.

Fecal incontinence is the inability to control your bowel movements, causing stool (feces) to leak unexpectedly from your rectum. Also called bowel incontinence, fecal incontinence ranges from an occasional leakage of stool while passing gas to a complete loss of bowel control in someone who is older than 4 years old. Common causes of fecal incontinence include constipation, diarrhea, and muscle or nerve damage. Fecal incontinence may be due to a weakened anal sphincter associated with aging or to damage to the nerves and muscles of the rectum and anus from giving birth.

Pudendal nerve (PN) entrapment (Alcock canal syndrome) is a further pathologic situation responsible for bladder disorders. Pudendal neuralgia is an uncommon source of chronic pelvic pain, in which the pudendal nerve is entrapped or compressed. Pain is located in the perineal, genital and perianal areas and is worsened by sitting. By simple entrapment of the PN without neurogenic damages, pain is usually isolated and can be associated with OAB. In neurogenic damage to the PN, genitor-anal numbness, fecal and/or urinary incontinence can occur. PN entrapment can be caused by obstetric traumas, scarring due to genitoanal surgeries (prolapse procedures), accidents and surgical mishaps. Sacral radiculopathies (sacral nerves roots S#2-4) are underestimated etiologies also frequently responsible for pudendal pain with irradiation in sacral dermatomes, bladder hypersensitivity or in neurogenic lesions, bladder retention.

Erectile dysfunction is an additional field of indication for PN stimulation. Erectile Dysfunction is often a result of a combination of psychological and organic factors, but it is thought to be purely psychological in origin in less than 30% of the cases. Organic factors can include complications from neurologic diseases (stroke, multiple sclerosis, Alzheimer's disease, brain or spinal pathologies), chronic renal failure, prostate pathologies, diabetes but first of all pelvic surgeries and medications. However, most cases of erectile dysfunction are associated with vascular diseases. An erection cannot be sustained without sufficient blood flow into and entrapment within the erectile bodies of the penis, and vascular related erectile dysfunctions can be due to a malfunction of either the arterial or the venous system.

Various treatment modalities for urinary function disorders have been developed. The modalities typically involve drugs, surgery, bladder infiltration, or combinations.

Pharmacotherapy appears to moderate the incidence of UI episodes, but not eliminate them.

Current treatments for OAB include medication (anticholinergica), diet modification, programs in bladder training, detrusor infiltration with botulinum toxin A, but also surgery and electrical stimulation. Limitations of medical treatment may be limited efficacy over time, but first of all side effects such as dry mouth, dry eyes, dry vagina, blurred vision, cardiac side effects, such as palpitations and arrhythmia, drowsiness, urinary retention, weight gain, hypertension and constipation, which have proven difficult for some individuals to tolerate.

One present surgical modality for treatment of incontinence as well as urgencies involves the posterior installation by a percutaneous needle of electrodes through the muscles and ligaments over the S3 spinal foramen near the right or left sacral nerve roots (Interstim® Treatment, Medtronic). The electrodes are connected to a remote neurostimulator pulse generator implanted in a subcutaneous pocket on the right hip to provide unilateral spinal nerve stimulation. This surgical procedure near the spine is complex and requires the skills of specialized medical personnel. In terms of outcomes, the modality has demonstrated limited effectiveness. For people suffering from urinary urge incontinence, less than 50% have remained dry following the surgical procedure. In terms of frequency of incontinence episodes, less than 67% of people undergoing this procedure reduced the number of voids by greater than 50%, and less than 69% reduced the number of voids to normal levels (4 to 7 per day). This modality has also demonstrated limited reliability. 52% of people undergoing this procedure have experienced therapy-related adverse events, and of these 54% required hospitalization or surgery to resolve the issue. 33% require surgical revisions. It has also been reported that 64% of people undergoing sacral nerve neuromodulation for urinary incontinence are not satisfied with their current treatment modality (National Association for Incontinence, 1988).

In combinations of urinary and faecal disorders, because sacral nerve stimulation does not permit stimulation and/or neuromodulation of all pudendal fibers, it is difficult to treat urinary and faecal disorders with the same effectiveness.

Another proposed alternative surgical modality (Advanced Bionics Corporation) entails the implantation through a 12 gauge hypodermic needle of an integrated neurostimulator and bipolar electrode assembly (called the BION® System) through the perineum into tissue near the pudendal nerve of the left side adjacent the ischial spine. The clinical effectiveness of this modality has not been proved; the main problem is high rate of migration of the implant away from the pudendal nerves, with risk of migration being increased by sitting position, gluteal muscle activation and in women, sexual activities.

Another proposed alternative surgical modality consists of the bilateral stimulation of both branches of the dorsal genital nerves using a single lead implanted in adipose or other tissue in the region at or near the pubic symphysis (Benett et al—US 2007/0239224). This technique of implantation below the pelvis without any protection of the electrode by anatomical structures exposes the patient to migration (dislocation), disconnection or breakage of the electrode and/or the lead. Furthermore, this technique is too restrictive since it enables only treatment of urinary dysfunctions but not faecal dysfunctions or all pelvic pain situations (vulvodynia, pudendal neuralgia, etc.).

Methods and tools for implanting electrodes into the human body are known in general from the prior art. In this general context, it is assumed to be known in particular to implant electrode wires, that is to say elongate wire-shaped conductors having a contact face at one end and at the other end a connection for a signal generation source, at or in the direct vicinity of a nerve in the human body in order to apply to nerves or nerve ends electrical signals generated by means of the signal generation source in order to stimulate said nerves or nerve ends.

The applicant has therefore developed laparoscopic surgical technology, known by the name LION, with which electrode wires can be implanted in a therapeutically particularly effective manner into an inner pelvic region or pelvic floor of a patient so as to feed there the stimulation signals in a stimulating manner to the pelvic nerves, in particular the nerve ends or nerve roots. To implement this technology, it is known to use an endoscope, wherein a working channel provided on the endoscope lead is used as a shaft to implant the provided electrode wire under visual control by means of the endoscope (with endoscope head guided suitably to the position of implantation and with suitably surgically prepared position of implantation at the nerve or pelvic nerve root).

A technology of this type, assumed to be category-defining, is complicated however in terms of handling and implementation: not only are considerable demands placed on the surgical knowledge or surgical capability of the operator in question, but the substantially parallel alignment, required by the known technology, between optical observation axis on the one hand (the visual control or controllability by means of the endoscope) and the feed of the electrode wire through the endoscopic working shaft on the other hand is also unfavorable for exact alignment and positioning specifically of the critical nerve contact portion at the end of the electrode wire. In other words, simple and reliably positioned handling of the electrode wire at the site of implantation in the human inner pelvic region under optical control of the endoscope is impeded especially with orthogonally running geometries, which further increases the demands placed on the operator.

A further problem with this device known from the prior art lies in the fact that, with a wire electrode implanted via the working channel of an endoscope, said wire electrode (once the endoscope has been removed, whereby the electrode is then left at the site of implantation) protrudes via its connection portion opposite the nerve contact portion from the bodily access point used for the endoscope (typically arranged in the abdominal region, for example the navel). In order to then connect this connection portion of the electrode wire to a signal generation source (which typically is also implanted beneath the patient's skin), it is necessary to lay or surgically pass the connection-side end of the electrode wire in the superficial bodily region, which additionally increases the complexity of the procedure and subjects the patient to further potential stress.

From US 2007/0198065 A1 filed by the applicant, it is known to provide neurostimulation of nerves (e.g. plexus sacralis, nervus ischiadicus, nervus pudendus) in the small pelvis by using collector electrodes with eight ring-shaped outer segment electrodes that are spaced axially apart from one another, which collector electrodes are usually implanted by laparoscopy. The collector electrode is connected by a connection cable to a pacemaker, which acts on the collector electrode with a stimulation pattern and in doing so controls the outer segment electrodes individually, in order to selectively stimulate the desired nerve. In the implantation of the pacemaker, it is often necessary for the operator to pull the connection cable in the direction of the axial end having the collector electrode. It can happen that the operator pulls the connection cable in the direction of the pacemaker instead of in the direction of the collector electrode and thus shifts the latter away from the nerve to be stimulated, which then entails awkward repositioning of the collector electrode.

US 2009/0248124 A1 discloses a non-wire-shaped collector electrode for implantation in the human body, wherein the connection cable of the electrode is provided, within a short axial section, with a marker so as to be able to identify the electrode and/or to assign the electrode to the correct connection channel of the pacemaker. In the known electrode, the problem of directional orientation does not arise, since the comparatively broad electrode cannot be shifted out of position by pulling on the connection cable.

US 2010/0030298 A1 discloses an implantable electrode having a marker with which the rotary orientation of the connection for contacting a pacemaker can be determined.

Based upon the foregoing, it is clear that there is a certain need for an improvement in implantation of leads and electrodes for treatment of a wide range of afflictions.

### SUMMARY OF THE INVENTION

The main aspect of the invention provides a system and method for treating conditions such as urologic and/or faecal dysfunctions by stimulation of the endopelvic portion of the pudendal nerve (PN) or of the sacral nerves roots (S2,S3,S4).

An additional application of the invention is in using PN stimulation for treatment of refractory or neurogenic pudendal neuralgia by stimulation of the pudendal aferrent fibers contained in the nerve itself.

A further additional advantage of PN stimulation is an improvement of erectile function. Stimulation of the pudendal nerve afferents activates spinal circuitry that coordinates efferent activity in the cavernous nerve, increasing filling via dilatation of penile arteries, and efferent activity in the PN, preventing leakage via occlusion of penile veins, producing a sustained reflex erection.

As an additional advantage, the inventive method and system of implantation for stimulation of the sacral nerve roots and the sciatic nerve can be used for treatment of refractory sacral radiculopathies (and all kinds of pain syndromes induced by sacral radiculopathies such as coccygodynia, vulvodynia, vaginal pain, etc.), sciatica and all neuropathic pain situations in the lower extremities (sciatica, Sudeck Morbus, mononeuropathies, phantom pain/stump pain, etc.).

One aspect of the invention provides systems and methods for the treatment of pelvic floor disorders such as urologic dysfunctions, faecal dysfunctions and sexual dysfunction by the stimulation of the supralevator portion of the pudendal nerve (endopelvic portion). The invention is based on a simple, easy, safe and reproducible technique using a tunneling/applicator tool for implantation of an electrode lead to the endopelvic portion of the PN under laparoscopic control.

In one embodiment, the system and method will stimulate specifically and directly the sensory fibers of the PN that has a consistent inhibitory effect on reflex bladder and rectum contraction as well as on pudendal pain. This differs from other electrical stimulation approaches to treat urinary and faecal incontinence, which apply electrical stimulation to the sacral nerve roots or to the dorsal genital nerves alone or to the infralevator portion of the pudendal nerve.

Another aspect of the invention provides systems and methods for treating urologic dysfunctions. The systems and methods include laparascopically forming a first entry through the abdomen; introducing an applicator assembly through a second entry, the applicator assembly comprising a flexible introducer sleeve and a curved applicator tool disposed in the sleeve; manipulating a proximal end of the curved applicator tool to position a distal end of the curved applicator tool at an identified exposed nerve; and placing an electrode lead through the applicator assembly to the nerve.

The lead is introduced to the transpelveo area abdominally, under endoscopic vision, with placing of the electrode being done using a tunneling/applicator tool so that the electrode is in direct contact with the PN under the sacrospinous ligament. The site of implantation can first be exposed by laparoscopic surgery and simple detachment of pelvic lymph-fett-tissue from the pelvic side wall, exposing in this way the PN in anatomic planes.

The form of the tunneling/applicator tool offers a safe and quick placement of the electrode to the PN while avoiding dissection of the nerve itself and without need of transection of the sacrospinous ligament.

The pelvic dysfunctions to be treated can include urinary and/or fecal incontinence, micturition/retention, defecation/constipation, neurogenic and non-neurogenic overactive bladder, sexual dysfunctions, pelvic floor muscle activity and spasms/spasticity, neurogenic and non-neurogenic detrusor-sphincter-dyssynergia, and pelvic pain, especially pudendal pain, vulvodynia and ano-rectodynie. The methods according to the invention can be indicated in women, men and children (for example with spina bifida or other malformations of the uro-intestinal-genital tract, or neurologic malformations).

In a another embodiment, the system and method can be used to stimulate specifically and directly the sensory fibers of the sacral nerve roots, and or the sciatic nerves in their endopelvic portion, that has a consistent inhibitory effect on all neuropathic pain from the lower extremities, the pelvic floor and the pelvic organs.

Creating a small incision in the lower abdomen or using a laparoscopic trocar incision may further include advancing a sleeve and a curved tunneling/applicator tool first transpelveo-abdominally to a retroperitoneal position, then:

by following the external aspect of the peritoneum of the pelveo-abdominal sidewall to the previously dissected retroperitoneal obturator space and finally to the sciatic nerve and/or the PN by passing dorsally to the sacrospinous ligament.

by entering the previously dissected pararectal space and after transection of the sacral hypogastric fascia, placement of the lead electrode perpendicularly to the sacral nerve roots, that enable stimulation of all sacral nerves roots together or in different combinations with only one lead.

The lead is sized and configured to be implanted by passing through the mentioned sleeve with different lengths varying between 30 cm and 60 cm, depending on the anatomy of the patient. The distal portion of the lead includes flexible expandable anchoring structure that deploys from a collapsed condition after removal of the sleeve. The anchoring structure secures the distal portion of the lead in direct contact to the nerve and prevents dislodgement and/or migration of the electrode. Further flexible anchoring structures may be placed about 10-20 cm proximally of the distal anchoring structures (circumferentially spaced-apart, radiating tines, for example). These structures also deploy after removal of the sleeve and resist dislodgement and/or migration of the electrically conductive portion within the retroperiteal space below the abdominal fascia of the pelveo-abdominal wall.

The distal anchoring structures are distal to the distal most electrode and are desirably sized and configured to permit the electrode position to be adjusted easily during insertion, allowing placement at the optimal location in direct contact to the nerve. The proximal anchoring structure or means functions to hold the electrode at the implanted location despite motion of the tissue of the pelveo-abdominal wall and small forces transmitted by the lead due to relative motion of the connected pulse generator due to changes in body posture or external forces applied to the pelveo-abdomen. However, the anchoring means are also configured to allow reliable release of the electrode at higher force levels, to permit withdrawal of the implanted electrode by purposeful pulling on the lead at such higher force levels, without breaking or leaving fragments, should removal of the implanted electrode be desired.

Anchoring means can take the form of an array of shovel-like paddles or scallops. The paddles are desirably present as relatively large, generally planar surfaces, and are placed in multiple rows axially. The paddles may also be somewhat arcuate as well, or a combination of arcuate and planar surfaces. A row of paddles comprises two paddles spaced degrees apart. The paddles may have an axial spacing between rows of paddles in the range of six to fourteen millimeters, with the most distal row of paddles, and each row may be spaced apart 90 degrees. The paddles are normally biased toward a radially outward condition, where they will project into tissue. In this condition, the large surface area and orientation of the paddles allows the lead to resist dislodgement or migration of the electrode.

Desirably, the anchoring means is prevented from fully engaging body tissue until after the electrode has been deployed. The electrode is not deployed until after it has been correctly located during the implantation process and the sleeve has been removed. With the sleeve in place, the paddles are held in a collapsed condition against the lead body within the sleeve. In this condition, the paddles are shielded from contact with tissue. Once the desired location for the electrode is found, the sleeve can be withdrawn, holding the lead and electrode stationary. Free of the sleeve, the proximal and the distal paddles spring open to assume their radially deployed condition in tissue, fixing the electrode twice at the nerve and in the pelveo-abdominal wall retroperitoneally below the fascia. In the radially deployed condition, the paddles have a diameter, fully opened, of about four millimeters to about six millimeters, and desirably about 4.8 millimeters.

The paddles are not stiff, i.e., they are generally pliant, and can be deflected toward a distal direction in response to exerting a pulling force on the lead at the threshold axial force level, which is greater than expected day-to-day axial forces. The paddles are sized and configured to yield during proximal passage through tissue in response to such forces, causing minimal tissue trauma, and without breaking or leaving fragments, despite the possible presence of some degree of tissue in-growth. This feature permits the withdrawal of the implanted electrode, if desired, by purposeful pulling on the lead at the higher axial force level.

The proximal portion of the lead also preferably includes at least one visual marker that indicates the distal and proximal direction of the lead to make the removal of an extension cable easier when a two-stage procedure has been planned.

The implantation can be done unilaterally or bilaterally using the same laparoscopic approach during the same surgical time.

Another aspect of the invention provides a method comprising providing a stimulation electrode assembly comprising an elongated lead sized and configured to be implanted in adipose tissue, the lead including an electrically conductive portion to apply electrical stimulation to nerve tissue innervating.

Another aspect of the invention provides a curved tunneling/applicator tool that passes through the sleeve, with at least two removal tips (screws), one stump for implantation of the lead to the nerve, and one sharp for tunneling the lead or an extension cable (in two-stage procedure) subcutaneously in adipose tissue from the pelveo-abdominal wall.

An aspect of the invention may also include providing a sleeve having an interior bore sized and configured to create percutaneous transpelveo-abdominal access, and implanting the electrically conductive portion and at least one expandable anchoring structure in the selected region includes passing the electrically conductive portion and at least one expandable anchoring structure through the interior bore of the sleeve, the interior bore of the sleeve retaining the expandable anchoring structure in the collapsed condition to accommodate passage of the electrically conductive portion and the expandable anchoring structure through the portion and the expandable anchoring structure through the interior bore into the selected tissue region. The expandable anchoring structure may be normally biased toward the expanded condition.

Another aspect of the invention may include providing an implantable pulse generator sized and configured to be positioned subcutaneous to a tissue surface in an anterior pelveo-abdominal region remote from the at least one electrically conductive surface, and coupling the implantable pulse generator to the stimulation electrode assembly, wherein conveying electrical stimulation (low/high frequency, noise current) includes operating the implantable pulse generator to convey electrical stimulation through the stimulation electrode assembly to achieve selective stimulation of the PN. Programming and/or interrogating the implantable pulse generator using transcutaneous communication circuitry and recharge of the pulse generator from outside the body may also be included.

Based upon the foregoing, a method is provided in accordance with the invention for implanting an electrode to an endopelvic portion of a pelvic nerve, which method comprises the steps of laparascopically forming a first entry through the abdomen; introducing an applicator assembly through a second entry, the applicator assembly comprising a flexible introducer sleeve and a curved applicator tool disposed in the sleeve; manipulating a proximal end of the curved applicator tool to position a distal end of the curved applicator tool at an identified exposed nerve; and placing an electrode lead through the applicator assembly to the nerve.

In further accordance with the invention, an apparatus is provided for implanting an electrode to an endopelvic portion of a pelvic nerve, which apparatus comprises a flexible introducer sleeve; and a rigid curved applicator tool disposed in the sleeve.

An object of the present invention is therefore to create a device and a system with which the medically therapeutically proven and extremely beneficial implantation of wire electrodes can be simplified, in particular in the inner pelvic region or pelvic floor region of the human body, so that even less experienced operators can simultaneously reliably implant nerve contact portions at pelvic nerves or nerve roots in a positionally accurate manner, even with nerve geometries running at an angle to an endoscopic direction of observation. At the same time, a device and a system are to be created, whereby reliable and precise electrode implantation can be implemented in a minimally invasive manner and with low traumatic or injury risk at the site of implantation and when feeding the electrode to the site of implantation. Lastly, the problem of providing an easier option for laying and contacting the electrode wire at the end opposite the nerve contact portion, in particular the problem of providing the electrode wire already such that it can be contacted at its connection portion with a signal generator (which more preferably is likewise to be implanted) with little effort, reliably and without the need for complex intracorporeal laying procedures, is to be solved.

The surgical application tool in the combination according to the invention, having a rod and sleeve fitted or guided thereover, advantageously firstly makes it possible to reach the desired position of implantation in the inner pelvic region by guiding the tool extracorporeally through the lower pelvic region of the patient and then along the interior of the pelvis (more specifically the pelvic inner wall) as far as the pelvic nerves. The present invention, with the application tool introduced into the body and following the removal of the rod (that is to say with the sleeve remaining in place and providing a guide through the sleeve interior for the electrode wire now to be inserted from outside the body), thus makes it possible to reach all relevant pelvic nerves or the roots thereof located in the interior of the pelvis. These nerves include the relatively superficial nerves, for example the lumbar plexus, femoral nerve, the ilioinguinal nerve, genitofemoral nerve, lateral cutaneous nerve of thigh or iliohypogastric nerve. By means of the application tool according to the invention, the deeper pelvic nerves can equally be reached, such as the sacral plexus, the sciatic nerve, the femoral nerve, the splanchnic pelvic nerves, the pudendal nerve or the levator ani nerve, the superior hypogastric plexus and the inferior hypogastric plexus.

A further aspect of the invention is to provide a collector electrode which has a connection cable, and with which an inadvertent shifting of the collector electrode away from the nerve to be stimulated can be reliably avoided during a surgical intervention. The system comprising a correspondingly improved collector electrode which is connected by the connection cable to a pacemaker for applying a stimulation pattern to the collector electrode is also advantageously combined with the electrode anchoring system described above.

In an implantable collector electrode of the type in question, particularly a collector electrode implantable by laparoscopy, this object is achieved by the fact that a direction marker perceptible by sight and/or by touch is provided on the outer surface of the connection cable, at least in an axial section which is spaced apart from the axial ends of the connection cable, said direction marker indicating the orientation of the connection cable to the operator.

As regards the system, the object is achieved by the combination of a collector electrode, designed according to the concept of the invention, together with a pacemaker.

Advantageous additional developments of the invention are also set forth herein. The scope of the invention covers all combinations of at least two of the features disclosed in the description, the claims and/or the figures.

In order to avoid repetition, features that are disclosed in relation to the device are also to be understood as having been disclosed and able to be claimed in relation to the method. Likewise, features that are disclosed in relation to the method are to be understood as having been disclosed and able to be claimed in relation to the device.

The invention is based on the concept of avoiding inadvertent shifting of the collector electrode during implantation of the pacemaker, by providing a direction marker, perceptible by sight and/or by touch, on the outer surface of the connection cable, at least in an axial section which is spaced apart from the axial ends of the connection cable, said direction marker indicating the orientation of the connection cable to the operator. The orientation of the connection cable is to be understood as the direction in which, on the basis of the direction marker, the collector electrode or the axial end directed away from the collector electrode is located. The direction marker thus has the function of providing the operator with information concerning the orientation of the connection cable, such that the operator can see, from the axial section observed, in which direction the connection cable is to be pulled, without this resulting in undesired shifting of the collector electrode at the end away from the nerve. The feature “at least in an axial section which is spaced apart from the axial ends of the connection cable” is to be understood as meaning that the orientation information is intended to be recognizable to the operator in an area spaced apart from the axial ends. Of course, the axial section having the direction marker can also extend as far as at least one of the two axial ends of the connection cable, although the direction marker must at least also be provided in an area spaced apart from the ends. It is important that the operator obtains the orientation information without having to see one of the two axial ends, particularly since these ends are located in regions of the body that are not visible, that are concealed or that are not exposed.

The direction marker can be designed to be perceptible by sight, for example by suitable printing on the outer surface. If appropriate, the marker can also be designed to be perceptible by touch. A direction marker perceptible by touch can be obtained by a raised and/or recessed formation of the direction marker.

The invention is based on the concept that the direction marker is provided over a (relatively long) cable section of at least 10 cm in length and/or of at least 15% of the total length of the connection cable and is designed in such a way that every 2 cm, preferably every 1.5 cm, more preferably every 1.0 cm, very particularly preferably every 0.5 cm, of this cable section is enough to allow the operator to visually establish the orientation of the connection cable (without further optical aids such as magnifying lenses). To put it another way, provision is made according to the invention that the axial extent of the cable section with direction marker measures at least 10 cm, preferably at least 15 cm, more preferably at least 20 cm, very particularly preferably at least 25 cm, and that the orientation of the connection cable can be read off at any desired section of this at least 10 cm long cable section which (the section) has a length of at most 2 cm, preferably at most 1.5 cm, more preferably 1.0 cm, very particularly preferably 0.5 cm. Of course, the preselected section of the cable section provided with the direction marker can also be larger. However, according to the invention, it is sufficient to have an axial extent of 2 cm or less, very particularly preferably of 0.5 cm or less.

The solution according to the invention is therefore in two steps. First, the cable section with the direction marker must be sufficiently long (according to the invention at least 10 cm and/or 15% of the total length of the connection cable), and, second, the direction marker must be so configured, for example by a suitable number of visual markers per centimeter, that the orientation can be read off at each section of this cable section if the section has a longitudinal extent of 2 cm (or more), preferably 1.5 cm (or more), preferably 1 cm or more, very particularly preferably 0.5 cm (or more). With a typical connection cable length of 60 cm, 15% of the total longitudinal extent corresponds to approximately the at least 10 cm.

In the case where the axial extent of the cable section provided with the direction marker is smaller than the axial extent (total axial extent) of the connection cable, it is particularly expedient if most (more than 50%, preferably more than 60%) of the axial extent of the cable section with direction marker is then situated in the proximal section (toward the pacemaker) of the connection cable.

The total axial extent of the connection cable is understood as the axial extent between the collector electrode at the end and the contact or connection area for contacting the pacemaker.

As has already been mentioned, the collector electrode designed according to the concept of the invention serves for neurostimulation of endopelvic nerve sections in the small pelvis, by means of a pacemaker applying a defined stimulation pattern to the collector electrode or the outer segment electrodes of the collector electrode. The stimulation pattern is preferably chosen according to the indication that is to be treated. The collector electrode designed according to the concept of the invention can alternatively also be used as a sensor for detecting nerve impulses. The collector electrode designed according to the concept of the invention is preferably suitable for use in the following indications:

Neurogenic or non-neurogenic hyperactivity of the bladder (sacral and pudendal nerve stimulation);

Neurogenic or non-neurogenic, myogenic hypotonia/atonia of the bladder;

Pudendal block (in paraplegia);

Voiding of the bladder/bowels in hyperactivity deblockade (in paraplegia);

Spasticity in the lower extremities, particularly in multiple sclerosis, polyneuropathy, quadriplegia/paraplegia, etc. (sciatic nerve stimulation);

Erectile and sexual problems, loss of erection, inability to ejaculate, premature ejaculation (stimulation of sciatic nerve root/stimulation of pudendal nerve);

Inability to achieve orgasm in females;

Neurogenic and non-non-neurogenic urinary/rectal incontinence (stimulation of pudendal nerve);

Chronic constipation;

Various pathologies and symptoms (simultaneous stimulation of various (at least two) nerves); and

Pudendal neuralgia=>sciatic nerve neuralgia.

In addition to restoration of bladder and/or bowel function, deambulation, in particular by stimulation of the sciatic nerve (cf. illustrative embodiment according to FIG. 5) or of at least one sacral nerve root or all the sacral nerve roots (cf. illustrative embodiment according to FIG. 6), the collector electrode designed according to the concept of the invention is suitable for use in further indications listed below:

Therapy of neuropathic pain of the lower extremities (Sudeck's syndrome, stump and phantom pain after amputation, polyneuropathy), in particular through stimulation of the afferent fibers of the sciatic nerve.

Control of spasticity of the lower extremities, in particular from spinal cord injuries or multiple sclerosis.

Muscle build-up in the lower extremities, particularly in the buttocks for prevention of decubitus ulcers in paraplegic patients. Here, the approach followed is to generate as much tissue as possible in an area between the wheelchair and the bone, in order to minimize decubitus ulcers. Muscle contraction is indicated by stimulation of the efferent fibers of the sciatic nerve, as a result of which muscle mass and strength are built up. By stimulation of the sympathetic fibers of the sciatic nerve, a peripheral vasodilation can. This not only serves to prevent decubitus ulcers but can also be used to treat decubitus ulcers.

The treatment of blood pressure problems in paraplegic patients, particularly in quadriplegics. To this end, it is advantageous to stimulate the sciatic nerve for the purpose of controlling the blood control of the lower extremities, which influence the general blood pressure.

Therapy of osteoporosis of the lower extremities in paraplegic patients. The blood supply to the bones is improved by stimulation of the sciatic nerve, particularly of the sympathetic fibers of the nerve. This serves on the one hand to prevent osteoporosis and also as therapy of osteoporosis.

The collector electrode is preferably arranged at an axial end of the connection cable or is formed by an axial end area of the connection cable. It is particularly preferable if the collector electrode has a wire-shaped design. Wire-shaped is to be understood here as an elongate, for example rod-shaped, rigid or, alternatively, possibly deformable configuration.

According to the invention, provision is made that the collector electrode has a wire-shaped design. Wire-shaped is to be understood here as an elongate, for example rod-shaped, rigid or, alternatively, possibly deformable configuration.

The axial extent of the in particular wire-shaped collector electrode is preferably chosen from a value range of between approximately 45 mm and approximately 65 mm. The axial extent is particularly preferably approximately 57 mm. The length dimension relates here to the distance between the opposite axial ends of the outer segment electrodes farthest from each other. It is particularly expedient if the diameter of the collector electrode, which is preferably at least approximately cylindrical, in particular circularly cylindrical, in contour is chosen from a value range of between 0.5 mm and 2 mm, very particularly preferably from a value range of between 0.8 mm and 1.2 mm. The diameter is still more preferably approximately 1 mm.

A very particularly preferred embodiment of the collector electrode is one in which the direction marker consisting of a multiplicity of individual symbols, or the cable section having the direction marker, has, in a section spaced apart by at least 10 cm, preferably at least 15 cm from the proximal end (toward the pacemaker), an axial extent of at least 10 cm, preferably at least 15 cm, more preferably at least 20 cm, very particularly preferably at least 25 cm, in order to be able to determine the orientation in an area of the connection cable spaced apart relatively far from the pacemaker, wherein at most every 2 cm, preferably at most every 1.5 cm, more preferably at most every 1.0 cm, very particularly preferably at most every 0.5 cm, of this cable section is enough to allow the orientation of the connection cable to be determined visually without optical aids.

As has already been indicated at the outset, the at least one axial section having the direction marker does not necessarily have to end at a distance from one or both axial ends of the connection cable, and instead, if so desired, it can be continued as far as at least one of the axial ends of the connection cable.

It is important, however, that the orientation of the connection cable can be read off from the direction marker at least in one area spaced apart from the axial ends.

In an alternative embodiment, the at least one axial section provided with a direction marker ends at an axial distance in front of at least one of the two axial ends of the connection cable.

This distance is preferably less than 25% of the total longitudinal extent of the connection cable, more preferably less than 15%.

There are various possibilities regarding the specific configuration of the direction marker. For example, it is possible that the direction marker is formed by a multiplicity of symbols that are arranged axially in succession, are spaced apart from one another or connected to one another, and are perceptible by sight and/or touch. For example, these can be arrow symbols that point in one of the two axial directions, in order thereby to indicate the position of the collector electrode or the position of the pacemaker relative to the arrow symbol. It is very particularly preferable if the symbols are arranged in a row. It is very particularly preferable if the symbols are spaced axially apart from one another. If necessary, several rows of symbols spaced apart in the circumferential direction can be provided. The at least one row of symbols preferably extends, at least more or less, along the entire axial extent of the connection cable.

It is essential to provide a sufficient number of symbols per unit of length, so as to ensure that the orientation can be read off at each section with a length of 2 cm, 1.5 cm, 1.0 cm, preferably 0.5 cm, of the at least 10 cm long cable section.

Particularly if no arrow symbols are used to indicate orientation, the orientation can be signaled by the fact that a geometric feature, for example an axial extent and/or a circumferential extent of the symbols, varies from symbol to symbol or from symbol group to symbol group, where each symbol group comprises at least two symbols. In other words, a geometric dimension, for example, decreases or increases from symbol to symbol toward one of the two axial ends, in order thereby to provide the orientation information upon simultaneous observation of at least two symbols or symbol groups.

In an alternative embodiment, particularly when arrow symbols are used, the symbols arranged one after another can be of identical design.

In another alternative embodiment, the direction marker can comprise a single symbol or a small number, e.g. only two, three or four in total, of axially adjacent symbols, wherein the information concerning direction or orientation can be read off from the change in a geometric dimension of the symbol or symbols. For example, the circumferential extent of the symbol can decrease toward one axial end, resulting, for example, in an extremely elongate arrow symbol.

A very particularly preferred embodiment of the collector electrode is one in which it comprises at least five, preferably at least six, very particularly preferably at least seven, still more preferably eight, outer segment electrodes that are spaced apart in the axial direction and are preferably ring-shaped.

An embodiment in which the collector electrode is arranged at one end on the connection cable is particularly expedient.

This means that the actual collector electrode, i.e. the arrangement of outer segment electrodes, closes off an axial end of the connection cable or forms an end section of the connection cable. Thus, the collector electrode is not situated at just any axial position on the connection cable, but expressly at an axial end, so as to be able to position the collector electrode optimally, in particular by grasping the connection cable. The collector electrode thus forms the end continuation of the connection cable or of the connection cable end, resulting in a wire-shaped configuration of the collector electrode/connection cable arrangement. The diameter of the connection cable preferably corresponds, at least more or less (±10%), to the diameter of the collector electrode preferably formed by the end section of the connection cable.

There are various possibilities regarding the configuration of the insulating sections and/or of the outer segment electrodes. For example, these can extend only around sections of the circumference. However, it is particularly preferable if the insulating sections and/or the outer segment electrodes are ring segments closed all the way round the circumference.

The axial extent of the outer segment electrodes is preferably chosen from a value range of between approximately 1 mm and approximately 5 mm. The axial extent is preferably approximately 3 mm. The axial extent of at least one of the insulating sections is preferably chosen from a value range of between 2 mm and 7 mm. The axial extent is preferably 3 mm or 6 mm.

The invention also leads to a system for neurostimulation of nerves, comprising a collector electrode as described above with connection cable, wherein the outer segment electrodes of the collector electrode can be electrically controlled individually and/or in groups by an in particular eight-channel pacemaker, wherein the pacemaker is arranged at an axial end of the connection cable directed away from the outer segment electrodes.

Other features and advantages of the inventions are set forth in the following specification and attached drawings.

### BRIEF DESCRIPTION OF THE DRAWINGS

A detailed description of preferred embodiments of the invention follows, with reference to the attached drawings, wherein:

FIG. 1 shows introduction of the applicator tool of the present invention through the pelveo-abdominal wall;

FIG. 2 shows placement of the applicator tool in position at the pudendal nerve by following the pelvic sidewall outside the iliac vessels;

FIG. 3 shows placement of the applicator tool in position to the sacral nerve roots;

FIG. 4 illustrates an applicator tool and sleeve in accordance with the invention;

FIG. 5 illustrates a stump tip attached to the applicator tool of the present invention;

FIG. 6 illustrates the sleeve component of the applicator of the present invention;

FIG. 7 illustrates the applicator tool in accordance with a preferred embodiment of the present invention;

FIG. 8 further illustrates two interchangeable tips which can be utilized with the applicator tool in accordance with the present invention;

FIGS. 9a and 9b illustrate two alternative configurations for the electrode lead in accordance with the present invention;

FIG. 10 illustrates a kit containing all necessary components of the system of the present invention;

FIG. 11 shows a system comprising a pacemaker connected by a connection cable to a collector electrode, wherein a row of arrow symbols arranged axially alongside one another is provided as direction marker on the connection cable,

FIG. 12 shows a system of analogous design to the system according to FIG. 11, with the difference that two rows of symbols spaced apart from each other in the circumferential direction are provided as direction marker,

FIG. 13 shows an alternative system comprising a collector electrode with connection cable and a pacemaker, wherein a repeating arrangement of two different symbols is provided as direction marker, and

FIG. 14 shows a system in which a multiplicity of symbols arranged one after another are provided as direction marker, which symbols are designed as ring elements in the illustrative embodiment shown, wherein the axial extent of the symbols decreases in the direction of the collector electrode.

### DESCRIPTION OF THE PREFERRED EMBODIMENTS

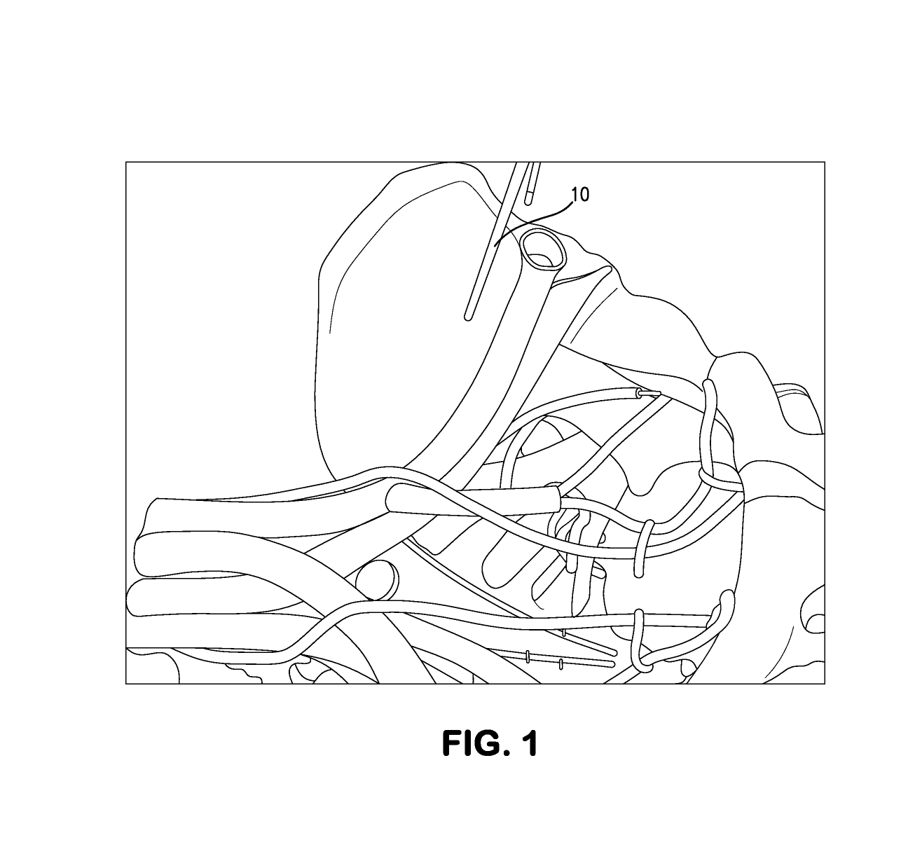

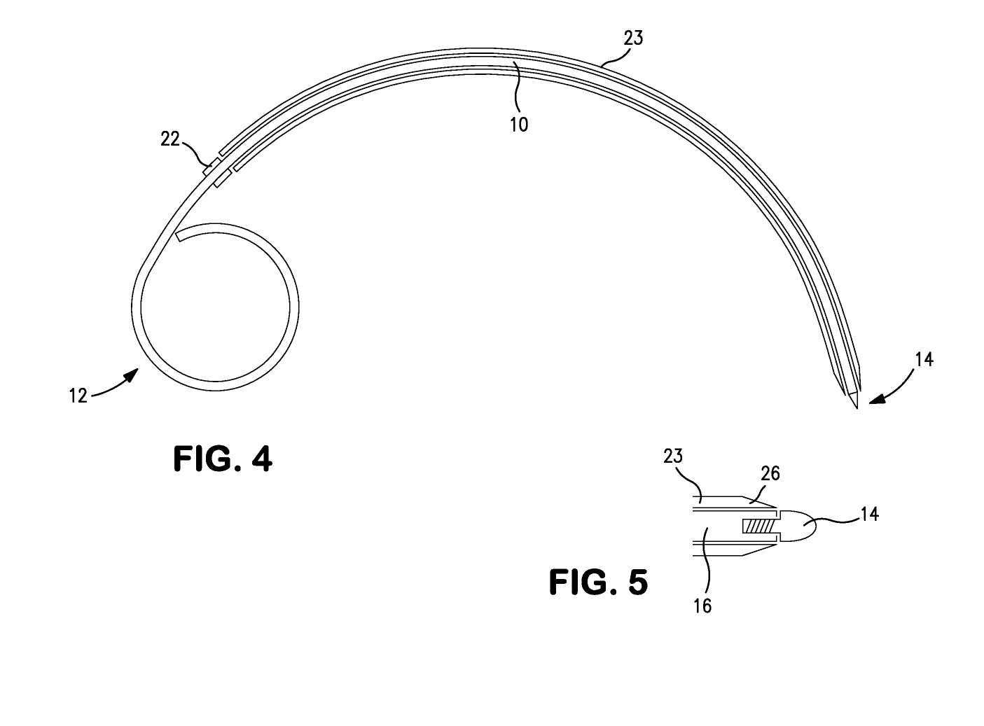

FIGS. 1-3 illustrate a model of the anatomical area of relevance to the present invention, with illustration of certain steps of the method of the present invention, while FIGS. 4-14 illustrate the tool and system of the present invention, all of which will be further described below.

I. System

A. The implant System

FIGS. 4-10 show an implant system for treating pelvic floor dysfunctions in humans.

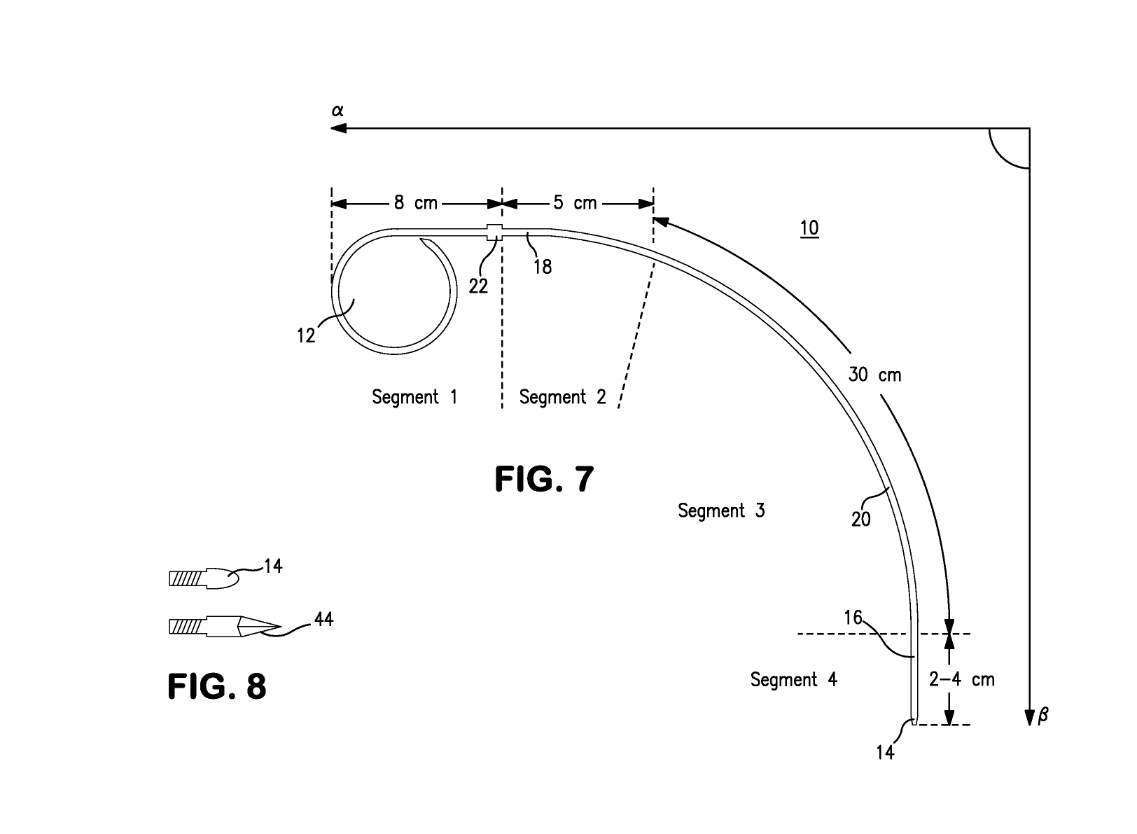

FIG. 4 shows a surgical system according to the invention which includes a curved tool 10 in a sleeve 23. Tool 10 has a handle portion 12 which can be curved or otherwise formed to be gripped by a surgeon or other user of the device. Tool 10 is further illustrated in FIG. 7. As shown, tool 10 can be formed from a cylindrical metal material and at one end forms curved grip portion 12 (segment A) and at the other end forms an engagement tip 14, which is formed at the end of a straight end or engagement segment 16 (segment 4, FIG. 7). Engagement tip 14 can be removable and replaceable as will be discussed below. A straight segment 18 with a length of approximately 5 cm and a curved segment 20, which may widen in terms of radius and then transition into the (distal) straight engagement segment 16 arranged at the end, are provided in this order between the grip portion 12, for which the rod metal having an outer diameter from 2 mm to 5 mm, in particular 2.5 mm to 3.5 mm, is curved in the shown manner to form a loop as a grip portion for extracorporeal access, and the engagement segment 16. A bending radius of the curved segment 20 varies between approximately 40 cm and approximately 80 cm in the direction of the distal end.

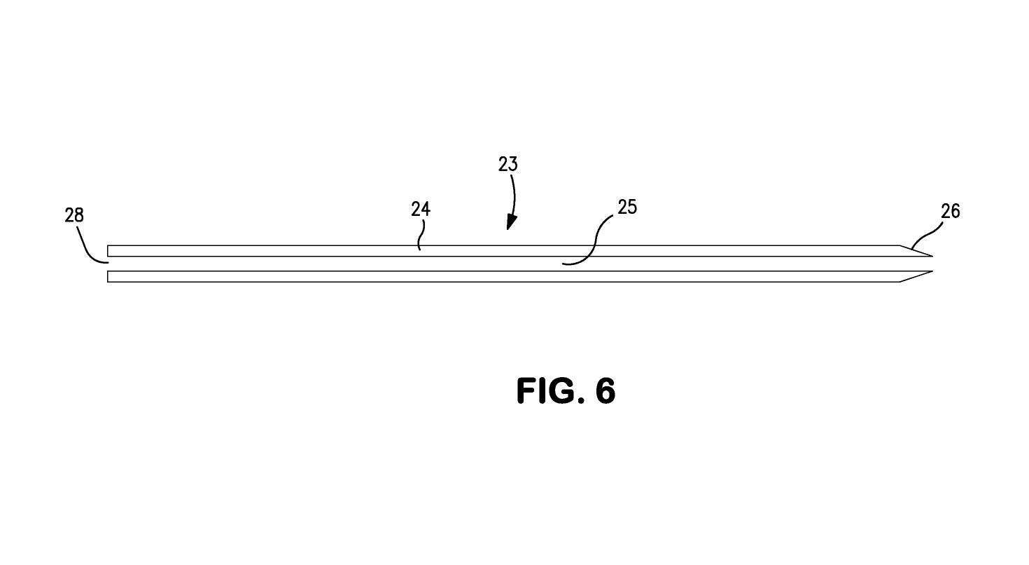

FIG. 6 shows sleeve 23 according to the invention, which can be formed from a flexurally rigid transparent plastic material, with a wall thickness of for example 2 mm. Sleeve 23 can be slid over the segments 18, 20, 16 of rod 10 and, supported by an abutment portion 22, can be brought, by engagement at the grip portion 12 and insertion into a bodily opening provided suitably in the region of the abdominal wall, into the body along the inner pelvic wall and as far as the pelvic floor or the pelvic nerves or nerve roots provided there. In an implanted state, the tip 14 would then mark the specific region in the interior of the pelvis at which the wire electrode (to be inserted later) can be placed with its nerve contact portion.

As illustrated in FIG. 6, the tubular elongate shaft region 24 of the sleeve 23 can be formed in a conically tapering manner in the direction of the distal end 26, wherein, in a preferred embodiment (see the illustration of the tip in FIG. 5 with the sleeve 23 assembled on the rod 10, a pointed cone of the sleeve 23 extends in a smooth conical course to and along the engagement tip 14, preferably continuously, such that in this respect, in this insertion configuration for the tool, there is no risk of tissue or vessel damage during the insertion process.

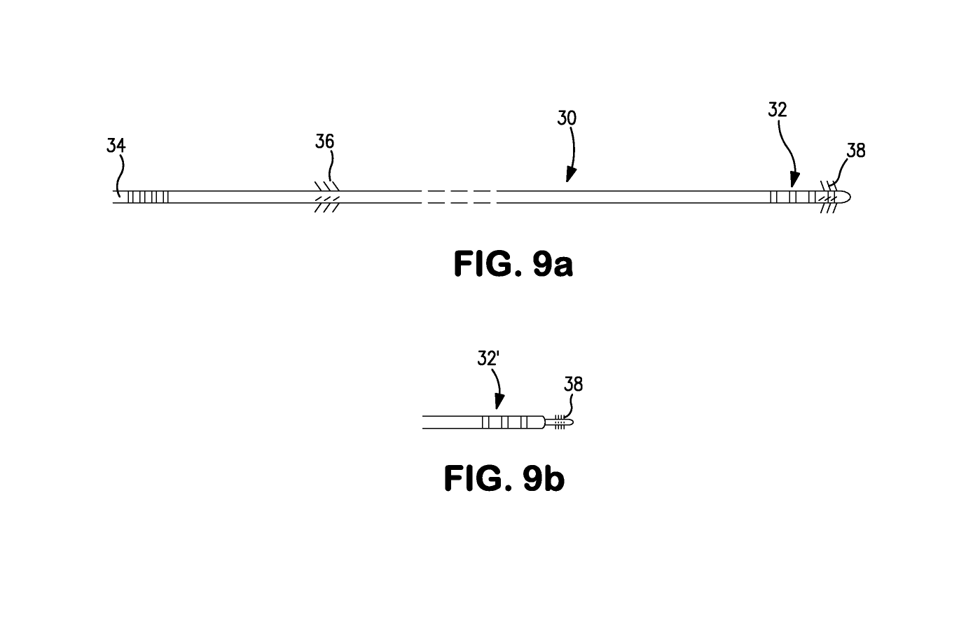

FIG. 4 in so far as it describes this insertion configuration, illustrates the fact that the resilient material of the sleeve 23 follows the straight and curved course (in segments) of the rod and in this respect provides a tool configuration that can be easily handled and positioned. Equally, the material of the lateral sleeve surface is designed such that it is smooth not only over the lateral surface (which is in turn favorable for friction-free and rupture-free sliding or advancing with insertion of the tool and movement of the tool in the body), and the material is also flexurally rigid in such a way that the shape of the rod (FIGS. 4 and 7) is still retained even when rod 10 is removed by being extracted once the site of implantation has been reached by engagement tip 14. Sleeve 23 remains in the body in this operational or operating stage (the geometries are typically selected such that, in the length portion of the sleeve corresponding to the segment 18, the sleeve exits from the body and, in an opening region 28 opposite the distal region 26, provides an insertion opening for an electrode wire 30 (FIGS. 9a and 9b) once rod 10 has been removed).

Specifically during use, electrode wire 30, for example having a typical length between 50 cm and 70 cm, would then be inserted via its distal nerve contact portion 32 (in this respect FIGS. 9a and 9b show two variants 32 and 32′ which will be further discussed below) into the sleeve 23 and guided through the hollow-cylindrical sleeve interior 25 to exit sleeve 23 at the desired location. During operation of the system according to the invention, the electrode wire is advanced via the distal end 32 or 32′ until it exits from the distal sleeve end 26, preferably under visual-optical control of an endoscope brought suitably via a separate bodily entrance to the site of implantation. Preferably, the position of the sleeve, into which it was brought by rod 10, remains unchanged after removal of rod 10 and insertion of lead 30, such that the nerve contact portion 32, 32′ is already at the intended nerve contact position (position of implantation) at the desired nerve. Where necessary, the surgeon has the option to undertake fine adjustments at the site of engagement under endoscopic control by means of minor manual actuation of sleeve 23 from the extracorporeal sleeve end 28.

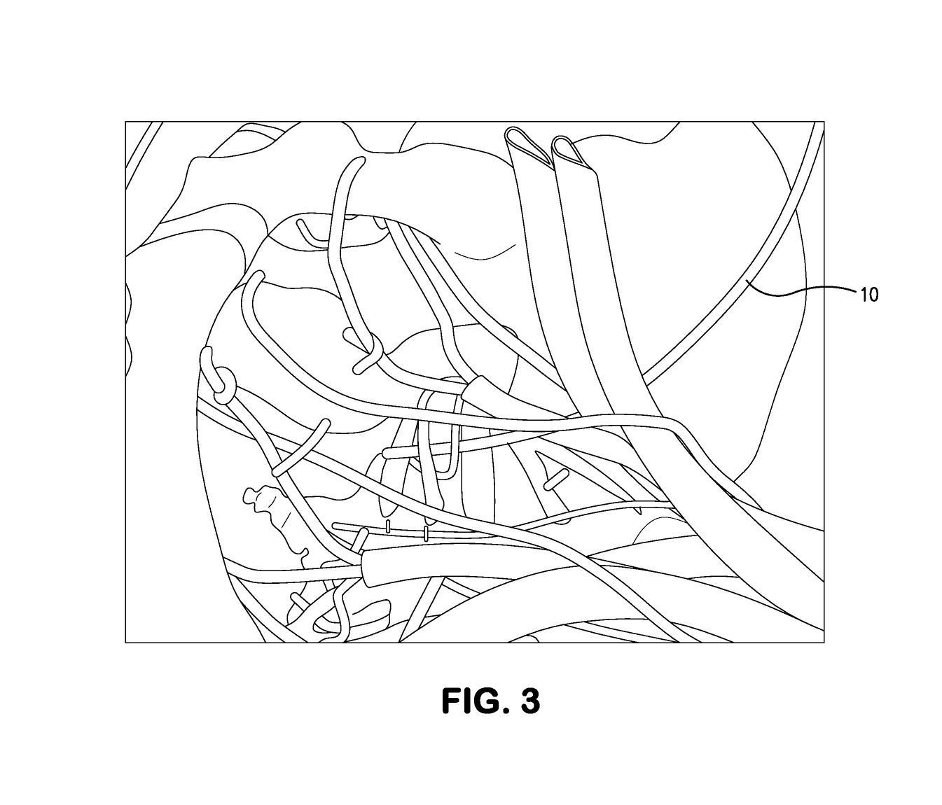

Referring to FIGS. 9a and 9b, lead 30 can comprise a multi-pole electrode having an outer diameter of approximately 1.8 mm and having 4 poles in the embodiment 32 shown in FIGS. 9a and 3 poles in the embodiment 32′ shown in FIG. 9b. The poles or contact portions 32, 32′ can be mechanically and electrically contacted at a connection portion 34 opposite the distal end (the location of poles 32, 32′) in a manner that is otherwise known, by means of peripheral electronics (for example a cardiac pacemaker electronics unit) or the like, wherein the respective contact portions 32, 32′ are guided via suitable strand structures in the interior of the wire electrode and can be contacted in the end region 34. In this manner, contact portions 32, 32′ are electrically connected to other components of the system of the present invention.

Still referring to FIGS. 9a and 9b, electrode wire 30 according to the invention can have barb means or locking means in the form of wings 36, which are arranged on the lateral surface, are directed radially in the direction of the proximal end 34, and which are arranged or fastened (preferably integrally) in a manner distributed around the periphery of the lateral surface of the wire in such a way that they bear closely against the lateral surface of the electrode during the displacement (sliding) in the sleeve interior 25 and in this respect enable an easy, low-force feed through sleeve 23. However, in an exposed state in the body once the electrode 30 has been placed in position and sleeve 23 removed, wings 36 implement a blocking effect with respect to tensile forces on the wire by radially expanding (spreading) and/or in the manner of a barb structure, said tensile forces being directed in the direction of the proximal end 34. In other words, the barb means or locking means 36 unfold in a wing-like manner in accordance with the invention and advantageously ensure that the wire electrode 30 is anchored in the body, such that bodily movements or an unintended traction on the electrode 30 does not cause an unwanted displacement or even extraction of the electrode from its site of engagement.

Barb portions 38, 38′ formed similarly in a wing-like manner can be provided at the distal end, either at a distal end of the structure on which contact portions 32 are formed (FIG. 9a) or on a narrower extension of the tip at distal end 32′ in the variant, with barbs 38′ formed in this tip region, such that a certain blocking effect or safeguarding against unintentional withdrawal is additionally and already offered from the moment at which the electrode 30 exits from the distal end of the sleeve 26. The distal anchoring means (barb means) 38 or 38′ specifically then also advantageously prevent the wire from being entrained for example as the sleeve 23 is manually removed, and once the electrode 30 has been inserted fully, and the wire remains in its desired implanted position, retains its predetermined implantation course (which is again determined by the predetermined curvature of the sleeve or the rod), and is ideally completely unaffected by the removal of the sleeve 23, such that, at the end of this operational step of the surgical application tool according to the invention, the wire electrode 30 remains in the body as the only implanted module.

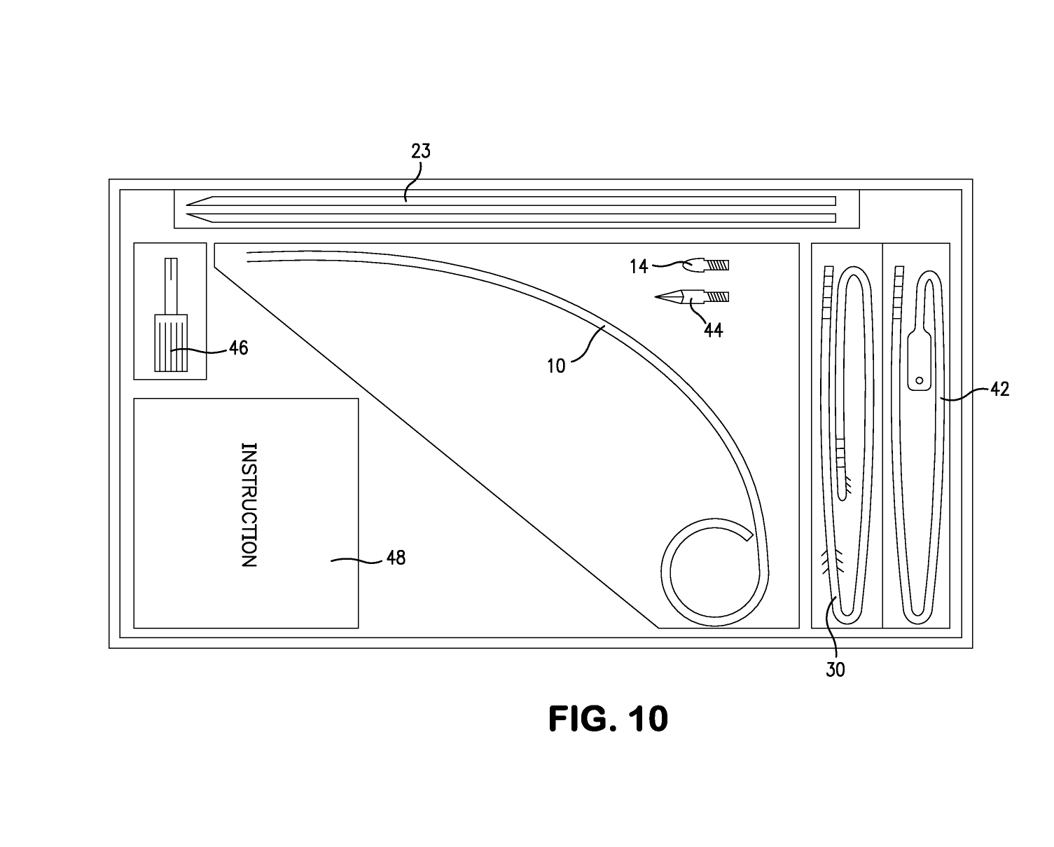

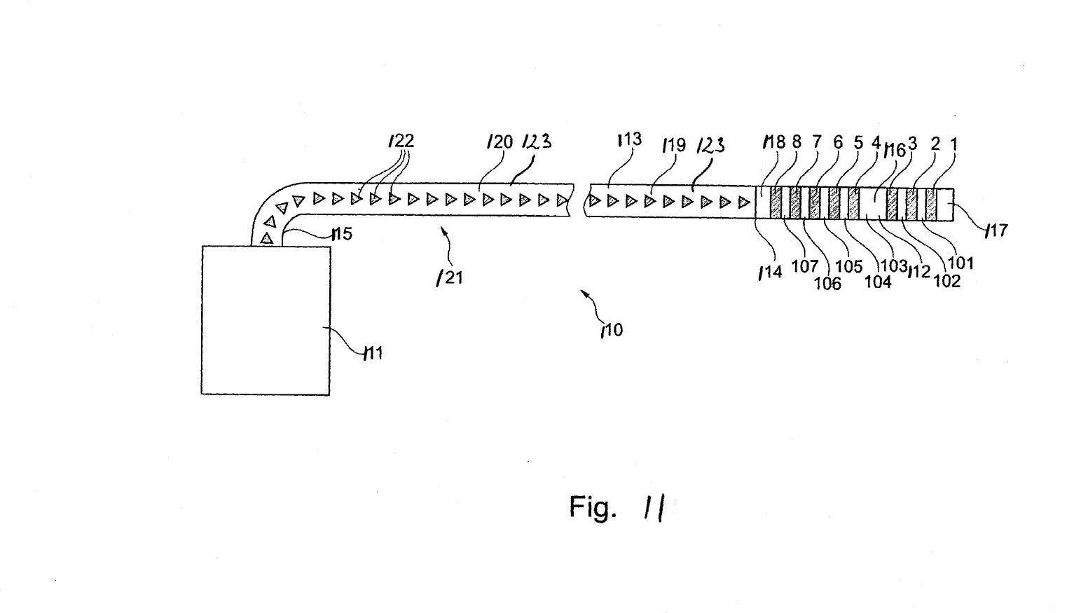

During further operation, either an electrode function test is then first performed via the contact-side, proximal end 34 of the implanted electrode 30 (via signal generation means connected extracorporeally) and suitable observation of the nerve response, or the pulse generator (not shown) would already be suitably connected, either in a manner connectable directly to the end 34 or by means of an additional possible connection wire 42 (FIG. 10), and then in turn placed suitably beneath the patient's skin; the advantageous extension 42 in accordance with a development, in conjunction with a (renewed) use of the surgical application tool consisting of the rod 10 and sleeve 23 for laying the extension wire 42 from the end position of the wire end 34 into another bodily position, enables greater versatility of the implantation. Further, the patient in question, at the opening necessary for the insertion of the application tool, experiences less stress or risk of infection on account of the signal generator to be implanted. In the ideal case, this bodily opening can be completely closed and can heal without further stress (with the exception then of the connection between the lines 30 and 42).

It is particularly favorable if the invention is provided in the manner shown schematically in FIG. 10 with the required components in the manner of an easily accessible package or kit. Besides the discussed main components of the rod 10 and sleeve 23, this kit also has an alternative engagement tip element 44 (See also FIGS. 8a and 8b) such that it can be exchanged by means of screwing or the like for a conically tapering, blunt element 14 (FIG. 8a). This is particularly suitable for forming the progression of the extension cable 42 (typically close to the skin in the abdominal region) in the optionally described second usage or treatment step for the extension cable.

As set forth above, end 34 carries a plug, which is desirably of an industry-standard size, for coupling to an industry-sized connector on a pulse generator. The distal end includes at least one electrically conductive surface, which will also in shorthand be called an electrode. The lead electrically connects the electrode itself, while electrically insulating the wire from body tissue except at the electrode.

The lead and electrode are sized and configured to be implanted percutaneously transpelveo-abdominally, and to be tolerated by an individual during extended use without pain or discomfort. The discomfort to be avoided is both in terms of the individual's sensory perception of the electrical waveforms that the electrode applies, as well as the individual's sensory perception of the physical or mechanical presence of the electrode and lead. In the case of the mechanical presence, the lead and electrode are desirably “imperceptible”.

Furthermore, the lead and electrode possess mechanical characteristics including mechanical compliance (flexibility) along their axis (axially), as well as perpendicular to their axis (radially), and are unable to transmit torque, to flexibly respond to dynamic stretching, bending, and crushing forces that can be encountered within soft, mobile adipose tissue in the pelveo-abdominal wall without damage or breakage, and to accommodate relative movement of the pulse generator coupled to the lead without imposing force or torque to the electrode which tends to dislodge the electrode.

The implantable lead comprises a molded or extruded component, which encapsulates one or more stranded or solid wire elements, and includes the connector. The wire element may be bifilar, and may be constructed of coiled MP35N nickel-cobalt wire or wires that have been coated in polyurethane. In a representative embodiment with two electrically conductive surfaces, one wire element is coupled to the distal electrode and the pin of the connector. A second wire element is coupled to the proximal electrode and possibly also the ring on the connector. The molded or extruded lead can have an outside diameter as small as about 1 mm, and desirably about 1.9 mm. The lead may also include an inner lumen having a diameter about 0.2 mm to about 0.5 mm, and desirably about 0.35 mm. The lead provides electrical continuity between the connector and the electrode.

A standard IS-1 or similar type connector at the proximal end provides electrical continuity and mechanical attachment to the pulse generator. The lead and connector all may include provisions for a guidewire that passes through these components and the length of the lead to the conductive electrode at the distal end.

The electrode may comprise one or more electrically conductive surfaces, and preferably 3 or 4 as shown in FIGS. 9a and 9b. The conductive surfaces can be used either as one or more individual stimulating electrodes (cathodic) in a monopolar configuration using the metal case of the pulse generator as the return (anodic) electrode or either the distal or proximal conductive surface as an individual stimulating (cathodic) electrode in a monopolar configuration using the metal case of the pulse generator (rechargeable or not) as the return (anodic) electrode or in bipolar configuration with one electrode functioning as the stimulating electrode (cathodic) and the other as the return electrode (anodic).

The electrode or electrically conductive surface or surfaces, can be formed from PtIr (platinum-iridium) or, alternatively, 316L stainless steel. Each electrode possesses a conductive surface of approximately 10 mm²-20 mm² and desirably about 16.5 mm². The surface area provides current densities up to 2 mA/mm² with per pulse charge densities less than about 0.5 μC/mm². These dimensions and materials deliver a charge safely within the stimulation levels supplied by the pulse generator.

Each conductive surface has an axial length in the range of about three to five millimeters in length and desirably about four millimeters. When two or more conductive surfaces are used, either in the monopolar or bipolar configurations as described, there will be an axial spacing between the conductive surfaces in the range of 1.5 to 2.5 millimeters, and desirably about two millimeters. The stimulation of the pudendal nerve includes normal usual stimulation/neuromodulation, high-frequencies stimulation, anode blockade or stimulation with noise.

It is appreciated that the term “stimulation” includes both excitation and inhibition or blocking of action potential in nerves (low/high-frequencies, noise, anodal blockade, etc.).

### B. Physician Surgical Tools

The implant system makes desirable a system of physician surgical tools to facilitate implantation of the implant system in the intended way, desirably on an outpatient basis.

The surgical tool system shown in FIG. 10 includes a curve tunneling/applicator tool 10 with two screwable tips, one sharp 14, one stump 44, and a companion introducer sleeve 23. The tunneling/applicator tool 10 can comprise a curved stainless steel shaft positioned inside introducer sleeve 23. The curve can start about two cm distal of the proximal end, and the last distal 3 cm can be straight for a parallel implantation of the lead to the pelvic nerves. The shaft, which may be bendable to allow adjustment for physical contours if required, includes handle 12 to aid the physician in delivering the tunneling tool to the desired location, and detachable screwable tip 14. The tunneling/applicator tool can be used with the stump tip 44 for implantation of the lead to the nerves avoiding this way vascular or nerve injuries. The tunneling/applicator tool is used with the sharp tip 14 to pass the implantable lead and extension cable (two-stage procedure) subcutaneously to the contralateral side (prevention of infection of the lead and electrode) and/or to the pulse generator pocket. The shaft of the tunneling/applicator tool and sleeve are about 15 cm to about 45 cm long (depending on anatomy of the patient), with the tip preferably extending less than 1 cm beyond the sleeve. The sleeve is also flexible to allow bending or curving and strong enough to avoid kinking of the sleeve itself after retraction of the steel shaft.

### C. Test-Screening Tools

In the above description, the surgical tool system allows an implant of the system in a single surgical procedure. Alternatively, and desirably, a two-stage surgical procedure can be used.

The invention comprises an intraoperative screening phase under urodynamic testing for evaluation of the stimulability of one or both PN or sacral nerves roots, and therefore to decide intraoperatively of an implantation unilaterally, or bilaterally.

The test screening system includes a percutaneous extension cable, which is sized and configured to be tunneled subcutaneously to a remote site where it exits the skin, usually located in the contralateral side of the pelveo-abdominal wall. The extension cable has a proximal and a distal portion. The proximal portion carries a standard female IS-1 receptacle for connection to the industry-standard size plug on the end of the electrode lead. The distal portion of the percutaneous extension cable carries a plug that is coupled (e.g. screws) to an external pulse generator. The components of a surgical tool system can be provided with the test screening system.

The extension cable also comprises a molded or extruded component, which encapsulates one or more stranded or solid wire elements, and electrically couples the receptacle and the plug. The wire element may be a solid or multifilament wire, and may be constructed of coiled MP35N nickel-cobalt wire or 316L stainless steel wires that have been coated in polyurethane or a fluoropolymer such as perfluoroalkoxy (PFA), or other wire configurations known in the art.

In a two-stage surgical procedure, the first stage comprises a screening phase of several weeks that performs test stimulation using a temporary external pulse generator to evaluate if an individual is a suitable candidate for extended placement of the implantable pulse generator. If the patient is a suitable candidate, the second stage can be scheduled, which is the disconnection and removal of the extension cable followed by the connection of the electrode-lead to the pulse generator and finally the implantation of the pulse generator itself in a subcutaneous pocket. For this surgical phase, the visual markers placed on the proximal portion of the lead indicate to the physician the distal and proximal direction of the lead that make the disconnection of the electrode-lead from the extension-lead safer and easier.

As FIG. 10 shows the various tools and devices as just described can be consolidated for use in a functional kit that can take various forms, and the arrangement and contents of the kit can vary. In the illustrated and preferred embodiment, the kit comprises a sterile, wrapped assembly of the components as shown and described above. The kit may be sterilized, for example using ethylene oxide. The kit includes an interior tray made, e.g., from die cut cardboard, plastic sheet, or thermo-formed plastic material, which holds the contents. The kit also preferably includes directions for using the contents of the kit to carry out a desired procedure or function.

The kit includes the lead electrode 30, the extension cable 42, a torque tool 46 (for screwing the electrode lead to the extension cable, and/or to the pulse generator), the tunneling/applicator tool 10, including the two different tips (sharp 14 and stump 44) and the sleeve 23, as well as instructions 48.

The directions or manual can of course vary. The directions shall be physically present in the kit, but also can be supplied separately. The directions can be embodied in separate instruction manuals, or in video or audio tapes, CDs and DVDs. The instruction for use can also be available through an internet page.

The technique of laparoscopic dissection of the interiliac space and exposure of the pelvic nerves including the technique of implantation can be embodied in separate manuals, or in video or audio tapes, CDs, and DVDs or can be available through an internet web page and/or learned during neuropelveologic courses and workshops designed for pelvic health care specialists such as surgeons, urologists and neurourologists, gynecologists and neurosurgeons.

II. Implanting the Implant System

### A. The Anatomic Landmarks

By way of background, the pudendal nerve is a sensory and somatic nerve which originates from the ventral rami of the second, third, and fourth (and occasionally the fifth) sacral nerve roots. After branching from the sacral plexus, the PN leaves the pelvis through the less sciatic foramen and travels to three main regions: the gluteal region, the pudendal canal, and the perineum. It accompanies the internal pudendal vessels upward and forward along the lateral wall of the ischiorectal fossa, being contained in a sheath of the obturator fascia termed the pudendal canal (Alcock's canal). The pudendal nerve gives off three distal branches, the inferior rectal nerve, the perineal nerve and the dorsal nerve of the penis in males, corresponding to the dorsal nerve of the clitoris in females.

The PN innervates the external genitalia of both sexes, as well as sphincters for the bladder and the rectum. As the bladder fills, the pudendal nerve becomes excited. Stimulation of the pudendal nerve results in contraction of the external urethral sphincter. Contraction of the external sphincter, coupled with that of the internal sphincter, maintains urethral pressure (resistance) higher than normal bladder pressure. The storage phase of the urinary bladder can be switched to the voiding phase either involuntarily (reflexively) or voluntarily. The pudendal nerve causes then relaxation of the levator ani so that the pelvic floor muscle relaxes. The pudendal nerve also signals the external sphincter to open. The sympathetic nerves send a message to the internal sphincter to relax and open, resulting in a lower urethral resistance. The PN is also known to have a potential modulative effect on bladder function. Somatic afferent fibers of the pudendal nerve are supposed to project on sympathetic thoracolumbar neurons to the bladder neck and modulate their function. This neuromodulative effect works exclusively at the spinal level and appears to be at least partly responsible for bladder neck competence and at least continence.

Stimulation of the PN provides direct and selective activation to the sensory fibers that lead to inhibition of the bladder and rectum and does not activate other nerve fibers that are present in the sacral nerves roots.

Stimulation of the PN provides direct and selective activation to the motoric fibers that lead to contraction of the anal and urethral sphincters to improve urinary and faecal incontinence without any activation of other nerve fibers that are present in the sacral nerves roots.

Stimulation of the pudendal nerve afferents activates spinal circuitry that coordinates efferent activity in the cavernous nerve, increasing filling via dilatation of penile arteries, and efferent activity in the PN, preventing leakage via occlusion of penile veins, producing a sustained reflex erection.

In a blind study of sacral versus pudendal stimulation for voiding dysfunctions, the majority of the patients chose the PN stimulation to be superior to sacral nerve stimulation (Peters K M et al. Neurourol Urodyn. 2005; 24(7):643-7).

Stimulation of the PN as an alternative to sacral nerve stimulation has been proposed in the past. However, the invasiveness of the surgical procedure for implanting leads made stimulation of the PN impractical. However, since the PN directly innervates much of the pelvic floor, it is believed to be a more optimal stimulation site with few undesired side effects. Implantation of electrode to the PN by laparoscopic approach can be done safely under control of endoscopic vision, is reproducible, performed in anatomic plane and uses anatomical landmarks and structures of which pelvic health care specialists are expert, as they commonly perform laparoscopic surgeries in the pelvic region.

Placement of the electrode in direct contact to the nerve (made possible by placement under direct observation through endoscopic vision) reduces risk for development of fibrotic tissue between the electrode and the nerves that could reduce the effectiveness of stimulation and consequently effectiveness of treatment.

Laparoscopic implantation can be done at the same surgical time and by the same surgical approach uni- or bilaterally.

The endoscopic transperitoneal or retroperitoneal approach for implantation the electrode avoids risk of injury to the spine associated with sacral nerve stimulation and risk of post operation hemorraghia or hematoma as by blind techniques of implantation. It does not require urodynamics, as simultaneous rectal palpation during intraoperative stimulation of PN is confirm by an evident contraction of the external anal sphincter by transanal digital palpation.

Minimally invasive surgery offers numerous potential benefits over conventional abdominal surgeries, including:

Shorter hospital stay, which can reduces costs.

Less pain, scarring and intrapelvic adhesions.

Less risk of wound infections.

Less blood loss and fewer transfusions.

Faster recovery and quicker return to normal activities.

Better preservation of immune system.

Despite advancement in lead anchoring techniques, the main problem of all techniques of implantation of leads outside the pelvic area is the high risk for lead migration, dislocation and cable brakeage. Endoscopic implantation of the electrode to the PN within the protection of the pelvic bone and above the pelvic floor protects from dislocation, disconnection and/or external trauma. Because in the deepness of the pelvis above the pelvic floor no movement occurs, because the electrode in the present invention is secure by distal and proximal tines and because the electrode is within the protection of the pelvic bone, there is practically no risk for electrode migration. This makes long term results of PN stimulation/neuromodulation better. The technique of laparoscopic transpelveo-abdominal access for implanting the lead electrode is to date the only technique that enables location of a lead electrode to the endopelvic portion of the pudendal nerve under control of vision.

### B. Implantation Methodology

Implantation of the implant system can entail a two-stage surgical procedure, including a test screening phase, or a single stage surgical procedure in which the pulse generator is implanted without a screening phase.

The first stage of implantation consists in the laparoscopic exposition of the nerves to which the electrode is to be implanted.

The laparoscopic step is performed under general anesthesia avoiding any myo-relaxation. The patients were given a single intraoperative antibiotic prophylaxis. For the trans- or retroperitoneal laparoscopy, one 10 mm trocar is placed in the umbilicus to introduce a 10 mm/0° optic and three additional 5 mm trocars are placed in the lower abdomen, one on the middle line and two lateral beyond the epigastric arteries to introduce an atraumatic forceps, scissors and bipolar forceps to control hemostasis. For intraoperative electrostimulation, a 5 mm bipolar laparoscopic forceps is used producing a current with square-wave pulse duration of 250 μs, a pulse frequency of 35 Hz, and an electric potential variable from 1 to 12 Volts. Single Port or Natural Orifice Approach can also be used for this surgery.

For the exposure of the endopelvic portion of the sciatic nerve and of the PN, the “lumbosacral way” or approach is used. After transection of the peritoneum laterally to the external iliac artery, exposure of the sciatic nerve is obtained by blunt dissection of the lumbosacral space along the psoas major and separation of the inter-iliac fatty-lymph-tissue from the obturatoric muscle laterally to the obturator nerve and vessels. Dissection or excision of the pelvic lymph nodes (that expose patients for risk for lymphocele) is not required since all the fett-lymph-tissue of the obturator space can simply be detached by blunt dissection from the internal obturator muscle (laterally to the obturator nerve and vessels) and retracted medially. By further exposure of the caudal border of the sciatic nerve, the endopelvic portion of the PN is identified. A dissection of the PN especially through the sciatic foramen is not necessary.

Confirmation of the functional integrity of the nerves is obtained by intraoperative laparoscopic stimulation of the nerve. PN stimulation even during the intervention induces a strong contraction of the external anal sphincter which can be confirmed by simple concomitant digital rectal examination.

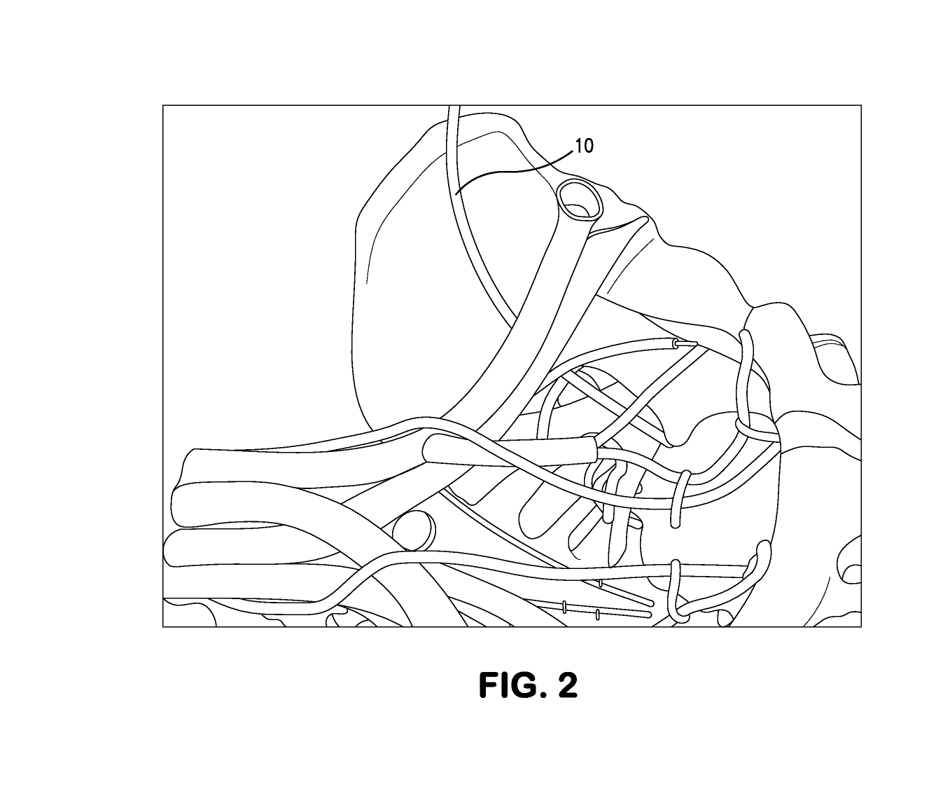

Once the location of the PN is found, the dissection is stopped. Next, the tunneling/applicator tool 10 with a stump tip and sleeve is introduced (a small 2-3 mm incision is required) just above the anterior iliac crest through the lateral abdominal wall until the top is identified intraabdominaly just below the peritoneum. FIG. 1 shows the subject anatomical structures and entry position of tool 10. By following the peritoneum and rotating the tunneling/applicator tool downwards, and thanks to the curve of this tool, it passes laterally from the external iliac artery into the previously dissected space. This step is absolutely safe since the top of the tunneling/applicator tool is permanently under control of endoscopic vision, that is, the insertion and positioning is conducted while being completely under observation through an endoscopic visual apparatus. Using the applicator tool, the electrode lead can be placed to the sciatic nerve, and/or the pudendal nerve. In implantation of the PN, the top of the tunneling/applicator tool is finally pushed under the sacrospinous ligament along the PN through the less sciatic foramen by about 1 cm. This position is illustrated in FIG. 2. Risk for lesion of the pudendal vessels is extremely minimal since the vessels are running on the opposite side of the PN.

Removal of the tunneling/applicator tool leaves the sleeve in place in direct contact to the nerve being implanted. This allows the physician to pass the electrode from outside through the sleeve to the nerve. After implantation to the nerve, the sleeve can be removed completely from the body, and toward the proximal end of the lead, that leaves the electrode in place and in direct contact to the nerve itself. The removal of the sleeve permits also the distal and proximal tines 38, 36 of the lead to deploy and to secure the location of the electrode twice, at the nerve (through the less sciatic foramen for the PN) and retroperitoneally in the adipose tissue from the abdominal wall below the abdominal fascia.

For exposure of the sacral nerve roots, the dissection is started by the incision of the pararectal peritoneum medial to the ureter and expansion of the anatomic pararectal space is carried out by absolute blunt dissection downwards to the level of the coccygeal bone. The dissection is expanded laterally to the hypogastric fascia which is transected in order to open the space lateral from it. The sacral roots S1 to S4 are selectively exposed by absolute gentle dissection and confirmation of the origin of the different sacral roots is gained by using laparoscopic electrostimulation—. LANN technique (Possover M, Rhiem K., Chiantera V. 2004. The “Laparoscopic Neuro-Navigation”—-LANN: from a functional cartography of the pelvic autonomous neurosystem to a new field of laparoscopic surgery. Min Invas Ther & Allied Technol 13: 362-367). Stimulation of S3 nerves is confirmed visually by a deepening and flattening of the buttock groove as well as a plantar flexion of the large toe and to a lesser extent of the smaller toes. Stimulation of S2 produces an outward rotation of the leg and plantar flexion of the foot as well as a clamp-like squeeze of the anal sphincter from anterior/posterior.

The lead electrode can then be placed easily by using the tool applicator while the lead is placed in between the sacral nerves roots and the pyriformis muscle. This placement protects the lead from dislocation and keeps the electrodes in direct contact to the nerves. This placement of the electrode is illustrated in FIG. 3.

The same technique using the applicator and a sleeve can also be used for implantation of “intelligent electrodes”, “integrated neurostimulator with electrode”, “anode blockade”, “implantable microstimulators”, any stimulation device with electronic circuitry for receiving data and/or power from outside the body by inductive, RF, or other electromagnetic coupling, implantable pump devices and other devices or implantable system, not only to the pudendal nerves but also to all other pelvic nerves (obturator nerve, femoral nerve, ilio-inguinal nerve, ilio-hypogastric nerve, lat. Cut. Femroralis nerve, genitofemroal nerve, etc.), nerve roots and plexuses (hypogastric plexis, sympathetic trunks, etc.) the laparoscopic transpelveo-abdominal way.

This technique of selective placement of the electrode without dissection of the nerves and necessity of extended dissection with transection of anatomic structures such as the sacrospinous ligament for PN implantation, makes the procedure safe, the operative time considerably shorter and the risk for migration of the electrode almost impossible.

Optionally, the test stimulator may be coupled to a lead electrode via a sterile cable to apply stimulation pulses trough the electrode, to confirm that the electrode resides in the location previously found.

If a test screening phase is planned, having implanted the lead electrode, a subcutaneous tunnel is formed for connecting the lead electrode to an extension cable. The same tunneling/applicator tool with a sharp tip and sleeve is introduced through the incision site where the lead electrode was passed transcutaneously, and pushed toward away from the primary incision to the contralateral side of the pelveo-abdominal wall. In this configuration, should infection occur in the region where the percutaneous extension cable extends from the skin, the infection occurs away from the region where the pocket for the implanted pulse generator is to be formed. The pocket incision site and the lead tunnel all the way to the electrode are thereby shielded from channel infection during the first stage, in anticipation of forming a sterile pocket for the implantable generator in the second stage.

If a one-stage procedure is planned, the lead can be connected directly to the generator that is placed in a subcutaneous prepared pocket.

### Direction Marking System

FIG. 11 shows a highly schematic further representation of a system 110 for neurostimulation of nerves. The system 110 comprises an implantable pacemaker 111, which is shown much too small in the drawing compared to a collector electrode 112 likewise included in the system. The pacemaker 111 is connected to the collector electrode 112 by a connection cable 113 which, as will be explained in more detail below, comprises a plurality of leads electrically insulated from one another. The collector electrode 112 adjoins the connection cable 113 axially, such that connection cable 113 and collector electrode 112 have a substantially wire-shaped configuration. The connection cable 113 comprises a first and distal end 114 at a transition to the collector electrode 112. Directed away from the distal and first axial end 114, the connection cable 113 has a second axial end 115, which adjoins the pacemaker 111.

In the preferred illustrative embodiment shown, the substantially cylindrical surface 116 of the collector electrode 112 comprises a total of eight outer segment electrodes 1 to 8, counting upward from the free end (situated on the right in the plane of the drawing) of the collector electrode 112. The outer segment electrodes 1 to 8 are individually controllable by means of the pacemaker 111 and are electrically insulated from one another. For this purpose, insulating sections 101 to 107 (likewise counting upward from the free end of the collector electrode 112) are situated between in each case two of the outer segment electrodes 1, 2; 2, 3; 3, 4; 4, 5; 5, 6; 6, 7; 7, 8 arranged axially one after another. It will be seen from FIG. 11 that the axial extent of the third insulating section 103, counting from the free end of the collector electrode 112, is greater and, in the illustrative embodiment shown, approximately twice as great as the axial extent of all the other insulating sections 101, 102 and 104 to 107. These other insulating sections 101, 102 and 104 to 107 have the same axial extent, approximately 3 mm in this illustrative embodiment. It will also be seen from FIG. 11 that the circumferentially closed outer segment electrodes 1 to 8, shaped as ring segments, are all of the same size, and they all have the same axial extent of 3 mm in the illustrative embodiment shown. The axial extent of the entire collector electrode 112 measures 57 mm in the illustrative embodiment shown. The diameter measures 1 mm.

It will also be seen from FIG. 11 that, to the right of the first outer segment electrode in the plane of the drawing, a first insulating end section 117 is provided, which is spaced apart from and faces away from a second end section 118 that forms the end directed toward the first end 114 of the connection electrode 113.

Alternatively, an embodiment is conceivable in which all the insulating sections 101 to 107 are of the same size. It is also possible that it is not the third insulating section 103, but another insulating section 101, 102 or 104 to 107, that is larger than the other insulating sections.

In the illustrative embodiment shown, the axial extent of the flexible, wire-shaped connection cable measures 20 mm. Each outer segment electrode 1 to 8 is individually contacted by an electrically insulated (control) lead, not shown for reasons of clarity, wherein all the leads are guided out from the collector electrode 112, specifically at the end of the collector electrode 112 directed toward the connection cable 113. Up to there, the leads are guided in the interior of the collector electrode 112 at a radial distance from the circumferential wall of the collector electrode. The leads join up to form the single connection cable 113 provided with a jacket 119 and used to contact the pacemaker 111.

The outer surface 120 of the connection cable 113 is provided with a direction marker 1x21, which indicates the orientation of the connection cable 113, that is to say the relative position of the axial ends 114, 115. In the illustrative embodiment shown, the direction marker 121 is formed by a multiplicity of in this case arrow-shaped symbols 122 arranged one after another in the axial direction, the tip of the arrows pointing in the direction of the collector electrode 112 in the illustrative embodiment shown. Alternatively, the tips of the arrows can of course be designed or arranged pointing in the direction of the pacemaker 111.

The operator simply has to know in which direction the arrow symbols point. In the illustrative embodiment shown, the symbols 122 arranged in a row extend, at least more or less, along the entire longitudinal extent of the connection cable 113 and are therefore also present in axial sections arranged at an axial distance from the ends 114, 115. The cable section provided with the direction marker 121 has a longitudinal extent of well over 10 cm and is indicated by reference sign 123. In the illustrative embodiment shown, the operator can read off the orientation of the connection cable 113 at any desired axial section of the cable section 123 having a length of at least 0.5 cm.

The following illustrative embodiments correspond substantially to the above-described illustrative embodiment of a system 110 as shown in FIG. 11, and therefore, in order to avoid repetition, it is essentially only the differences that will be discussed. As regards the common features, reference is made to the preceding figure description. In all of the illustrative embodiments, the cable section 123 with the direction marker is very long in relation to the total length of the connection cable, as is advantageous. In principle, it is sufficient for the cable section 29 to have a minimum length of 10 cm, preferably 15 cm, more preferably 20 cm. In all of the illustrative embodiments, which are not reproduced true to scale, it is ensured that the orientation can be read off at any desired 0.5 cm sections of the cable section 123.

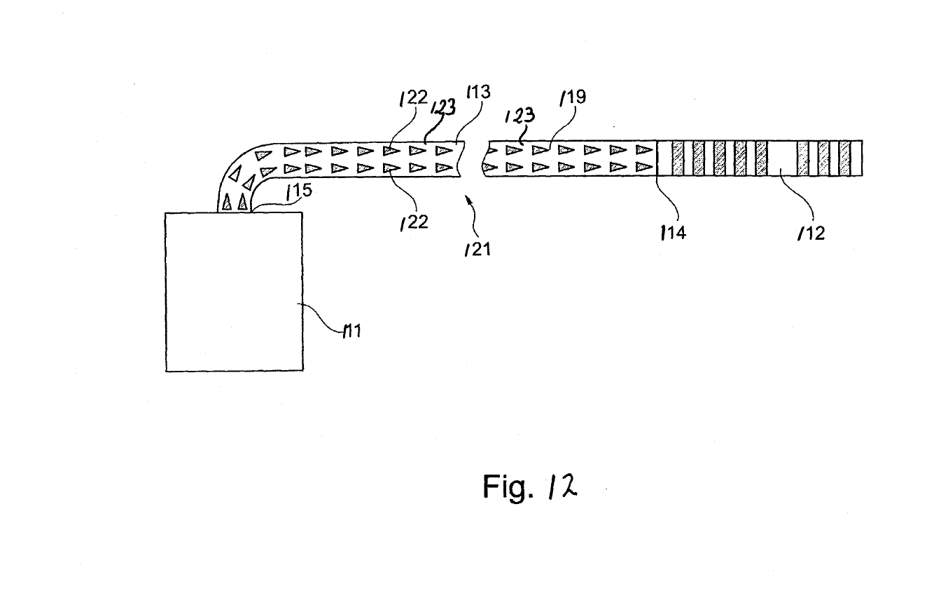

In FIG. 12, in contrast to the illustrative embodiment according to FIG. 11, the direction marker 121 comprises several rows of in this case arrow-shaped symbols 122 spaced apart from each other in the circumferential direction. As in the illustrative embodiment according to FIG. 11, the symbols 122 can be designed, for example, so as to be perceptible exclusively by sight.

It is preferable for them to be perceptible by a combination of sight and touch. For this purpose, the arrow symbols can be raised, for example, or designed as depressions in the jacket 119.

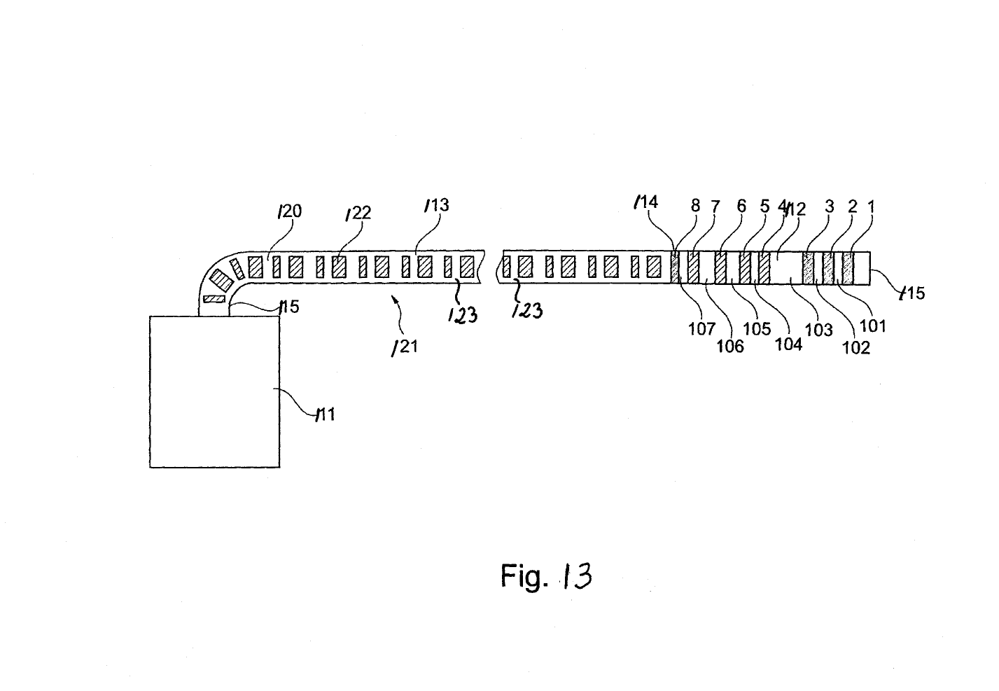

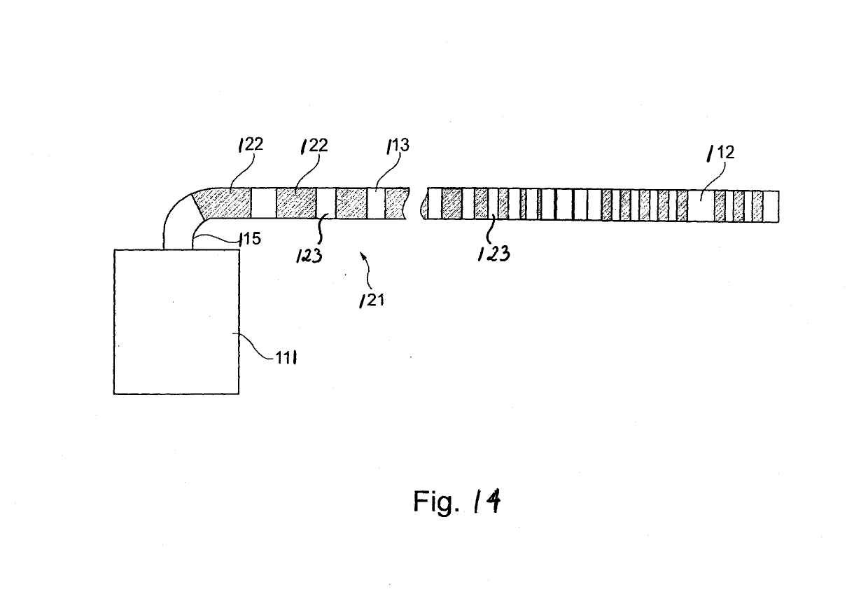

In the illustrative embodiment according to FIG. 13, the direction marker 121 on the outer surface 120 of the connection cable 113 comprises a repeated arrangement of symbols 122 which are each arranged in pairs and, through their arrangement relative to each other, embody directional information. In the illustrative embodiment shown, each pair of symbols comprises two rectangles of different sizes that differ in terms of their axial extent, wherein the distance between the symbols (small rectangle, large rectangle) of a symbol pair is different than the distance between in each case two adjacent symbol pairs. Instead of rectangles, other symbol geometries can also be chosen. Similarly, it is also possible for more than three contiguous symbols to embody directional information. In the illustrative embodiment shown, the distance between two symbols of a symbol pair is smaller than the distance between two symbol pairs. To be able to interpret the directional information, the user merely needs the information that, in the illustrative embodiment in question, the smaller symbols face in the direction of the pacemaker 111 and the larger symbols face in the direction of the collector electrode 112.

In the illustrative embodiment according to FIG. 13, an alternative preferred collector electrode 112 is provided that has a total of eight outer segment electrodes 1 to 8, wherein the insulating sections 101, 102 and 104 between the first and second outer segment electrodes 1, 2, between the second and third outer segment electrodes 2, 3 and between the fourth and fifth outer segment electrodes 4, 5 have a smaller axial extent, namely 3 mm in the illustrative embodiment shown, than the insulating sections 103, 105, 106 and 107 between the fourth and fifth outer segment electrodes 4, 5, between the fifth and sixth outer segment electrodes 5, 6, between the sixth and seventh outer segment electrodes 6, 7, and between the seventh and eighth outer segment electrodes 7, 8. The collector electrode shown in FIG. 13 can also be used with the alternative direction markers 121 of FIGS. 11, 12 and 14. If necessary, it is possible to omit the insulating section to the right of the first outer segment electrode 1 in the illustrative embodiment according to FIG. 13, and also in the other illustrative embodiments. The outer segment electrodes 1 to 8 and the insulating sections 101 to 107 are counted from the free end of the collector electrode 112.

The symbol pairs preferably extend over at least 25% of the total longitudinal extent of the connection cable 113.

In FIG. 14, the direction marker 121 comprises a multiplicity of successive symbols 122, which are spaced axially apart and, in the illustrative embodiment shown, are designed as ring-shaped symbols. The axial extent of the symbols 122 decreases from symbol to symbol in the direction of the collector electrode 112, such that the operator, by simultaneously observing two adjacent symbols 122, can read off the orientation of the connection cable 113.

All of the symbols shown in FIGS. 11 to 14 can be designed to be perceptible only by sight and/or to be perceptible by touch, for example by designing the symbols as elevations or depressions.

It should be appreciated that the foregoing is a description of preferred embodiments of the present invention, and that these embodiments are illustrated, but not limiting, upon the scope of the present invention. The scope of the invention, rather, is defined by the claims as appended hereto along with various modifications of parts, sizes and steps which would be readily apparent to a person of ordinary skill in the art.

1. A collector electrode assembly which can be implanted by laparoscopy through the abdominal wall into the small pelvis of the human body, comprising:
a collector electrode for neurostimulation of nerves;
a connection cable having an outer surface, said collector electrode being arranged at one end of said connection cable and comprising several outer segment electrodes which can be contacted individually and/or in groups and which are arranged axially one after another in the direction of the longitudinal extent of the collector electrode, wherein an insulating section is arranged axially between in each case two adjacent outer segment electrodes and permits electrical insulation of respective two adjacent outer segment electrodes;
radially expandable fixing structures positioned on the collector electrode and radially expandable from a withdrawn position to a radially expanded position for fixing the collector electrode in place at said nerves;
a visually perceptible direction marker on the outer surface of the connection cable, at least in a cable section which is spaced apart from axial ends of the connection cable and has an axial extent of at least 10 cm and/or at least 15% of total length of the connection cable, said direction marker indicating orientation of the connection cable to an operator using the assembly, and wherein the direction marker is designed and arranged in such a way that the identification of the orientation of the connection cable is possible at any desired axial section of the cable section having a maximum axial extent of 2 cm.

2. The assembly of claim 1, wherein the axial extent of the cable section measures at least 15 cm.

3. The assembly of claim 1, wherein the axial extent of the cable section measures at least 20 cm.

4. The assembly of claim 1, wherein the axial extent of the cable section measures at least 25 cm.

5. The assembly of claim 1, wherein the axial extent of the axial section provided with the direction marker is less than the total axial extent of the connection cable, and wherein more than 50% of the axial extent of the cable section is arranged between an axial connection cable center and the proximal end of the connection cable.

6. The assembly of claim 1, wherein the direction marker ends before at least one of the two axial ends of the connection cable at an axial distance whose longitudinal extent corresponds to 5% to 25% of the total longitudinal extent of the connection cable.

7. The assembly of claim 6, wherein the longitudinal extent of the axial distance is 5 to 15% of the total longitudinal extent of the connection cable.

8. The assembly of claim 1, wherein the direction marker comprises a multiplicity of symbols which are arranged axially one after another and are perceptible by sight and/or touch.

9. The assembly of claim 8, wherein the multiplicity of symbols are in the shape of directional arrows.

10. The assembly of claim 8, wherein a geometric feature of the symbols changes from symbol to symbol or from symbol group to symbol group as the distance to one of the axial ends of the connection cable decreases.

11. The assembly of claim 8, wherein at least two of the symbols are of identical design.

12. The assembly of 11, wherein the geometric feature is an axial extent and/or a circumferential extent of the symbols.

13. The assembly of claim 1, wherein the direction marker comprises at least one elongate symbol extending in the axial direction and changing in terms of geometry along its axial extent.

14. The assembly of claim 13, wherein the at least one elongate symbol extends over at least 25% of the longitudinal extent of the connection cable.

15. The assembly of claim 1, wherein the outer segment electrodes can be electrically controlled individually and/or in groups by an eight-channel pacemaker, and wherein the pacemaker is arranged on an axial end of the connection cable directed away from the outer segment electrodes.

16. The assembly of claim 1, wherein the desired axial section of the cable section has a maximum axial extent of 1.5 cm.

17. The assembly of claim 1, wherein the desired axial section of the cable section has a maximum axial extent of 1.0 cm.

18. The assembly of claim 1, wherein the desired axial section of the cable section has a maximum axial extent of 0.5 cm.

19. The assembly of claim 1, wherein more than 60% of the axial extent of the cable section is arranged between an axial connection cable center and the proximal end of the connection cable.
